(19) United States
(12) Patent Application Publication
(10) Pub. No.: US 2022/0375574 A1
(43) Pub. Date: Nov. 24, 2022

(54) SYSTEM AND METHOD FOR CALCULATING ACCURATE GROUND TRUTH RATES FROM UNRELIABLE SOURCES

(71) Applicant: Covera Health, New York, NY (US)

(72) Inventors: Tarmo Aijo, East Hampton, NY (US); Daniel Elgort, New York, NY (US); Xiaojin Dong, Berwyn, PA (US); Denis Whelan, New York, NY (US); Richard Herzog, New York, NY (US); Murray Becker, Belle Mead, NJ (US); Robert Epstein, Belle Mead, NJ (US); Irwin Keller, Belle Mead, NJ (US); Ron Vianu, New York, NY (US)

(21) Appl. No.: 17/736,890

(22) Filed: May 4, 2022

Related U.S. Application Data
(60) Provisional application No. 63/183,975, filed on May 4, 2021.

(51) Int. Cl. G16H 30/20; G06T 7/00

(52) U.S. Cl. CPC G16H 30/20; G06T 7/0012; G16H 30/40

(57) ABSTRACT

Described are techniques for determining an accurate ground-truth Bayesian Inter-Reviewer Agreement Rate (BIRAR) from unreliable sources. For instance, a process can include obtaining an initial set of diagnostic imaging exams, wherein each diagnostic imaging exam includes a severity grade associated with an initial radiologist. For each diagnostic imaging exam, two or more secondary quality assurance (QA) reviews can be obtained for each diagnostic imaging exam, wherein the secondary QA reviews are associated with QA'ing radiologists different than the initial radiologist. One or more inter-reviewer agreement rates can be determined for the QA'ing radiologists, based on the secondary QA reviews associated with the QA'ing radiologists. A diagnostic error associated with one or more initial radiologists can be determined based at least in part on the one or more inter-reviewer agreement rates for the QA'ing radiologists and a subsequent diagnostic imaging exam obtained for a respective one of the initial radiologists.

### CROSS-REFERENCE TO RELATED APPLICATIONS

This application claims the benefit of priority to U.S. Provisional Patent Application No. 63/183,975 filed May 4, 2021 and entitled “SYSTEM AND METHOD FOR CALCULATING ACCURATE GROUND TRUTH RATES FROM UNRELIABLE SOURCES,” the disclosure of which is herein incorporated by reference in its entirety.

### TECHNICAL FIELD

The present disclosure pertains to error detection and quantification, and more specifically pertains to improved accuracy in calculating an inferred ground truth rate from one or more unreliable sources, such as in a radiological quality assurance (QA) process.

### BACKGROUND

Radiology quality assurance (QA) programs often approach interpretive error detection by having radiologists perform secondary assessments of radiological studies that have been previously interpreted. This process is also commonly referred to as peer review and operates on the general principle that a lack of consensus between an original radiologist and a QA radiologist(s) likely indicates an interpretive or diagnostic error by the original radiologist. In other words, if an original read and a secondary read conflict, it is often assumed that the original read is incorrect. Such an approach may neglect to properly account for the error rate that is inherent in secondary reads as well. In many scenarios, a preferred approach for secondary review has not been validated (e.g., use of “experts,” a majority voting panel, non-expert single reads). Moreover, the discrepancy rates generated through secondary review have been shown to be unreliable measures of actual diagnostic error rates.

### SUMMARY

In some examples, systems and techniques are described for determining an accurate ground-truth Bayesian Inter-Reviewer Agreement Rate (BIRAR) from unreliable sources. According to at least one illustrative example, a method is provided, the method including: obtaining an initial set of diagnostic imaging exams, wherein each diagnostic imaging exam includes a severity grade associated with an initial radiologist; for each diagnostic imaging exam of the initial set, obtaining two or more secondary quality assurance (QA) reviews for each respective diagnostic imaging exam, wherein the secondary QA reviews are associated with one or more QA'ing radiologists different than the initial radiologist; determining one or more inter-reviewer agreement rates for the QA'ing radiologists, based at least in part on the secondary QA reviews associated with the QA'ing radiologists; and determining a diagnostic error associated with one or more initial radiologists, wherein the diagnostic error is determined based at least in part on the one or more inter-reviewer agreement rates for the QA'ing radiologists and a subsequent diagnostic imaging exam obtained for a respective one of the initial radiologists.

In another example, an apparatus is provided that includes a memory (e.g., configured to store data, such as virtual content data, one or more images, etc.) and one or more processors (e.g., implemented in circuitry) coupled to the memory. The one or more processors are configured to and can: obtain an initial set of diagnostic imaging exams, wherein each diagnostic imaging exam includes a severity grade associated with an initial radiologist; for each diagnostic imaging exam of the initial set, obtain two or more secondary quality assurance (QA) reviews for each respective diagnostic imaging exam, wherein the secondary QA reviews are associated with one or more QA'ing radiologists different than the initial radiologist; determine one or more inter-reviewer agreement rates for the QA'ing radiologists, based at least in part on the secondary QA reviews associated with the QA'ing radiologists; and determine a diagnostic error associated with one or more initial radiologists, wherein the diagnostic error is determined based at least in part on the one or more inter-reviewer agreement rates for the QA'ing radiologists and a subsequent diagnostic imaging exam obtained for a respective one of the initial radiologists.

In another example, a non-transitory computer-readable medium is provided that has stored thereon instructions that, when executed by one or more processors, cause the one or more processors to: obtain an initial set of diagnostic imaging exams, wherein each diagnostic imaging exam includes a severity grade associated with an initial radiologist; for each diagnostic imaging exam of the initial set, obtain two or more secondary quality assurance (QA) reviews for each respective diagnostic imaging exam, wherein the secondary QA reviews are associated with one or more QA'ing radiologists different than the initial radiologist; determine one or more inter-reviewer agreement rates for the QA'ing radiologists, based at least in part on the secondary QA reviews associated with the QA'ing radiologists; and determine a diagnostic error associated with one or more initial radiologists, wherein the diagnostic error is determined based at least in part on the one or more inter-reviewer agreement rates for the QA'ing radiologists and a subsequent diagnostic imaging exam obtained for a respective one of the initial radiologists.

This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

The foregoing, together with other features and embodiments, will become more apparent upon referring to the following specification, claims, and accompanying drawings.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended drawings. The use of a same reference numbers in different drawings indicates similar or identical items or features. Understanding that these drawings depict only exemplary embodiments of the disclosure and are not therefore to be considered to be limiting of its scope, the principles herein are described and explained with additional specificity and detail through the use of the accompanying drawings in which:

FIG. 1 depicts an example Error Probability Detection Matrix (EDPM), according to aspects of the present disclosure;

FIG. 2 depicts a Kullback-Leibler divergence (KLD) of the true distribution from the estimated distribution as a function of number of studies and number of reviews;

FIG. 3 depicts a schematic diagram of a quality assurance (QA) review process, according to aspects of the present disclosure;

FIG. 4 depicts box plots of the difference between the measured and actual diagnostic error rates of 100 QA'd radiologists using the five error rate measurement methods simulated in a study;

FIG. 5 depicts box plots of the difference between the measured and actual diagnostic error rates of 100 QA'd radiologists using the five error rate measurement methods simulated in a study;

FIG. 6 depicts box plots of the difference between the BIRAR measured and actual diagnostic error rates of 100 QA'd radiologists when using different volumes of QA'd Exams to calculate the EDPM;

FIG. 7A depicts box plots of a difference between measured and actual diagnostic error rates of 100 QA'd radiologists using five simulated error rate measurement methods, and more specifically illustrates results simulated under conditions where QA'ing radiologists have lower error rates than QA'd radiologists; and

FIG. 7B depicts results simulated under conditions where QA'ing radiologists have higher error rates than QA'd radiologists.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Additional features and advantages of the disclosure will be set forth in the description which follows, and in part will be obvious from the description, or can be learned by practice of the herein disclosed principles. It will be appreciated that for simplicity and clarity of illustration, where appropriate, reference numerals have been repeated among the different figures to indicate corresponding or analogous elements. The description is not to be considered as limiting the scope of the embodiments described herein.

### Overview

It would be desirable for radiology quality assurance (QA) programs (and more general QA programs as well) to incorporate one or more measurements of diagnostic error rates. For example, such a measurement of diagnostic error rate(s) would allow a QA program to both characterize and monitor important aspects of patient care quality, as well as to provide useful quality improvement feedback to radiologists. However, it remains challenging to measure and incorporate diagnostic error rate measurements into QA programs in a manner that is accurate, reliable, and scalable. For example, in a recently published study that surveyed radiologists' opinions of diagnostic error measurement and communication within QA programs, only 48.7% of the 291 participating radiologists reported that their “QA process is a process I trust.” [Lamoureux, “Radiologist Opinions of a Quality Assurance Program,” Academic Radiology, Vol 28, No 2, February 2021].

In certain contexts, accurate measurement of diagnostic errors is possible using a “gold standard” follow-up test that is performed subsequent to the radiology exam. These gold standard tests can be understood as explicitly or physically investigating the presence and extent of a pathology, whereas the radiology exam is typically understood as only implicitly or indirectly investigating the presence and extent of the pathology. For example, the accuracy of prostate MRI diagnoses can be measured through comparisons with transperineal prostate biopsies. However, relying on gold standard follow-up tests to enable measurement of radiology error rates within a QA program is neither viable nor practical for a number of reasons: (1) only a limited subset of radiology exam types and patient studies occur prior to gold standard diagnostic tests; (2) the gold standard follow-up test will generally only be performed after an initial positive radiology finding, which limits the ability for this approach to detect false negatives; and (3) the gold standard tests can themselves still suffer from errors, which reduces the reliability of any radiology error measurements derived on the basis of gold standard follow-up tests.

In light of the challenges associated with the use of gold standard follow-up tests to quantify diagnostic error rates, conventional and existing radiology QA programs typically rely upon a peer review process (also referred to herein as a “secondary review” or “secondary read”). Indeed, implementation of a secondary review process is a requirement for accreditation by the Joint Commission and the American College of Radiology (ACR). These peer review programs enable radiologists who perform secondary assessments of prior diagnostic imaging studies to flag discrepancies with the findings reported in the initial interpretation. Discrepancies flagged through these secondary assessments are often treated as measures of, or proxies for, diagnostic errors present in the initial interpretation of a radiological image.

However, these discrepancy rates have been shown to be unreliable measures of actual rates of diagnostic errors [Borgstede. “RADPEER Quality Assurance Program: A Multifacility Study of Interpretive Disagreement Rates” J Am Coll Radiol 2004; 1:59-65.] [McEnery “Comparison of Error Detection Rates in Mandatory vs. Voluntary Professional Peer Review” Proceedings of RSNA 2013.

To improve the accuracy and reliability of error rate measurements based on secondary reads (e.g., peer review assessments), some QA programs require multiple independent or consensus secondary assessments for a given, single initial review. For example, mammography QA programs have been reported which aim to quantify false negative cancer detection rates—mammography exams initially classified as negative are subjected to three independent secondary peer reviews; if at least two of three radiologists concur on the presence and location of cancer within the mammography exam, this is considered evidence of a false negative diagnostic error. [https://pubmed.ncbi.nlm.nih.gov/18540960/;DOI: 10.1111/j.1524-4741.2008.00593.x]. Conventional multiple secondary review/consensus approaches like these may yield more reliable measures of diagnostic error rates in comparison to peer review processes that rely on single secondary assessment, but the requirement for multiple secondary assessments or dedicated consensus discussions create significant demands on radiologists' time and resources (e.g., one impact being that the same fixed amount of secondary reviewing radiologist time is only sufficient to complete ⅓ as many secondary reviews in comparison to when using only a single secondary review QA process).

A central challenge associated with both radiology QA process, and broader QA processes as whole, is that it remains challenging to incorporate error rate measurements into quality assurance (QA) programs in a manner that is accurate, reliable, and scalable. With respect to the specific scenario of radiology QA, these elusive error rate measurements are more specifically in the form of diagnostic error rate measurements, both for individual radiologists and groups of radiologists (such as radiology practices). Accordingly, disclosed herein are systems and methods for a novel approach leveraging a Bayesian statistical framework to achieve accurate, reliable and scalable measurements of diagnostic error rates from peer reviews. Notably, the presently disclosed systems and methods for Bayesian Inter-Reviewer Agreement Rate (BIRAR) enable the determination of accurate error measurements without requiring a gold standard follow-up test or multiple assessments (e.g., secondary reads) of every exam evaluated by the BIRAR QA program.

BIRAR enhances and extends current quality assurance (QA) programs and confers several distinct advantages and improvements. According to aspects of the present disclosure, BIRAR permits the assessment of rates of radiologist diagnostic/interpretive error without requiring the review of cases/initial reads by experts; accounts for errors by the reviewing radiologist(s); allows for disagreement among reviewers; and provides an understanding of error rate accuracy with a minimum number of studies that need to be read by multiple reviewers.

Moreover, BIRAR enables diagnostic error rates to be measured accurately and is shown to be robust even when QA assessments are performed by radiologists who themselves have higher error rates than the QA'd radiologists—a significant improvement over conventional approaches to both QA error detection and the broader problem of calculating accurate ground truth rates from unreliable sources.

The discussion below begins with details of the BIRAR systems and methods that are the subject of the present disclosure. Following these technical details is a section summarizing the performance and results of a simulation study comparing BIRAR against four conventional methods for measuring diagnostic error rates. The BIRAR method is shown to be more accurate than all other methods evaluated, as quantified by median difference between measured and actual diagnostic error rates of the radiologists subject to QA review (with the exception of the hypothetical “Perfect QA-ing Radiologists” method, which is not possible in the real world given its assumption that the QA radiologists have a 0% error rate). Notably, BIRAR is demonstrated to be 80.3% more accurate than a “Majority Panel with 3× Total QA Review Volume” and additionally displayed lower accuracy variability than all other methods evaluated.

### Bayesian Inter-Reviewer Agreement Rate (BIRAR)

Disclosed herein are systems and methods for a novel approach for calculating accurate ground truth rates from unreliable sources. The following examples are made with reference to an example scenario involving a quality assurance (QA) process in which rates of diagnostic error are determined in an accurate, reliable, and scalable manner, leveraging a Bayesian statistical framework—however, it is appreciated that, without departing from the scope of the present disclosure, the presently disclosed systems and methods are applicable to a wider range of problems involving the calculation of accurate ground truth rates from unreliable sources (as will be discussed in greater depth in subsequent sections).

A novel approach termed BIRAR (Bayesian Inter-Reviewer Agreement Rate) is proposed for the measurement of diagnostic error rates. Notably, and advantageously, BIRAR does not require gold standard follow-up tests be compared with radiology findings, either initial reads or otherwise. Nor does the BIRAR-based approach require multiple secondary assessments to be performed for every radiology study or finding that is evaluated by the QA program. Instead, multiple independent secondary reads are performed for an initial set of QA'd exams and then used to characterize the QA'ing radiologists. In particular, an inter-reviewer agreement analysis is performed, driving the generation of one or more Error Detection Probability Matrices (EDPMs) either for the QA'ing radiologists as a whole, individual QA'ing radiologists, groups/subsets of QA'ing radiologists, or some combination of the three. Once the inter-reviewer agreement analysis has been performed, and the EDPM(s) generated, the BIRAR QA process can proceed with only a single secondary read for subsequent QA'd exams.

In particular, the BIRAR approach leverages a Bayesian statistical framework and involves two primary steps:
1) The first step characterizes the inter-reviewer agreement rates among the set of radiologists that are performing secondary assessments in a given QA program. The inter-reviewer agreement rates are then used to derive estimates of the probabilities that various types of diagnostic errors exist in a given QA'd exam, given that a discrepancy was flagged by a QA'ing radiologist. (As used herein, “QA'ing radiologist” refers to those radiologists who provide secondary reads as part of the QA review process; “QA'd radiologist” refers to those radiologists whose initial/original reads are subject to the QA review process; “QA'd exam” refers to the radiology exam or image corresponding to both the original and secondary reads).
2) The second step then uses the estimated probabilities for the different diagnostic error types, along with new data generated through subsequent single QA assessments of a set of patient studies, to calculate diagnostic error rates of individual radiologist and/or groups of radiologists covered by the BIRAR QA program.

The presently disclosed BIRAR approach begins by selecting an initial set of diagnostic imaging exams and subjecting each exam of the initial set to multiple (e.g., three) independent QA reviews by QA'ing radiologists. In some embodiments, 300-500 (or a similar order of magnitude) diagnostic imaging exams can be sufficient to initialize the BIRAR approach by characterizing the error rates among the QA'ing radiologists, although it is appreciated that other numbers of initial studies can be utilized without departing from the scope of the present disclosure (see, e.g., sensitivity analysis evaluating the impact of using a different number of initial QA'd exams having three secondary reads each, presented in the simulation discussion section). In the context of the present example however, an initial set of 300 diagnostic exams each subjected to three independent secondary reads yields a data set consisting of 900 QA'd exams upon which the inter-reviewer agreement analysis is performed and the EDPM(s) generated.

Notably, the set of QA'ing radiologists selected to perform the initial set of multiple independent QA reviews does not need to include all of the radiologists (or all of the reviewing radiologists) associated with the radiology practice or radiologist group that is the subject of the BIRAR-based QA review. Additionally, of the set of selected QA'ing radiologists, each individual radiologist is not required to provide a secondary read for each of the 300 initial diagnostic imaging exams. Instead, the BIRAR initialization process seeks to obtain a representative sample of different combinations of multiple (e.g., three) radiologists who provide secondary reads.

From the initial set of diagnostic imaging exams and their corresponding multiple independent QA reviews, the QA'ing radiologists' Error Detection Probability Matrix (EDPM) is estimated. The EDPM characterizes the conditional probabilities that a diagnostic error of a specific type is present in a QA'd exam, given the presence of a specific discrepancy flagged by a QA'ing radiologist who performed a secondary read of the QA'd exam.

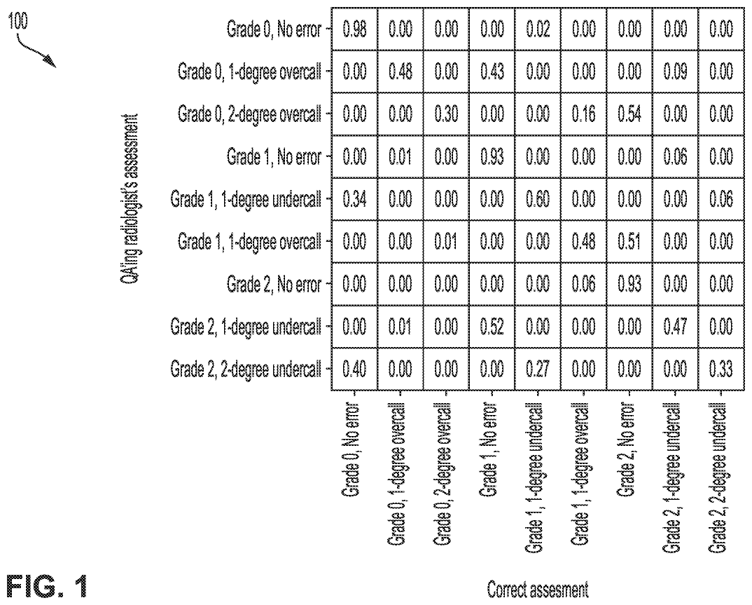

For purposes of illustration and context, an example EDPM 100 is depicted in FIG. 1. The following description makes reference to the example EDPM 100, although it is appreciated that other EDPMs and/or other EDPM conditional probability values can also be utilized without departing from the scope of the present disclosure. Recalling that the EDPM is an Error Detection Probability Matrix for the QA'ing radiologists, EDPM 100 can be generated to correspond to or otherwise be associated with a given one of the set of QA'ing radiologists. For example, EDPM 100 can be radiologist-specific, based on the given QA'ing radiologist's secondary reads that were provided in the aforementioned initial set of multiple independent QA reviews.

As illustrated, the matrix rows of example EDPM 100 indicate the QA'ing radiologist's assessment (e.g., provided in the QA'ing radiologist's secondary reads), while the matrix columns of example EDPM 100 indicate the correct (e.g., ground truth) assessment for the QA'd exam. The values within each box of the matrix represent the calculated probability associated with that specific row-column combination. The probabilities within each given row sum to one.

For example, the fifth row of the EDPM 100 of FIG. 1 corresponds to a QA'ing radiologist assessment of ‘Grade 1, 1-degree undercall’, meaning that the QA'ing radiologist determined that the pathology in a QA'd exam is Grade 1 and that the QA'd radiologist therefore committed a 1-degree undercall error (e.g., by reporting Grade 0). The values in the boxes of the fifth row indicate the following:
60% probability that the QA'ing radiologist finding of Grade 1 is correct; the QA'd radiologist finding of Grade 0 was incorrect (column five)
34% probability that the QA'ing radiologist finding of Grade 1 is incorrect; the QA'd radiologist finding of Grade 0 was actually correct (column one)
6% probability that both the QA'ing radiologist finding of Grade 1 and the QA'd radiologist finding of Grade 0 are incorrect; the correct finding for the QA'd exam is Grade 2 pathology (column nine)

### EDPM (Error Detection Probability Matrix): Calculation and Generation

With the above example of the resulting EDPM in mind, the disclosure turns now to the generation of the EDPM (e.g., EDPM 100), which can be understood in view of the following framework. For N_{grades} different pathology severity grades (e.g., 0, 1, 2), a set G of severity grade levels can be defined as:

G={0,1,2, . . . ,N_{grades}−1} Eq. (1)

By comparing the pathology grade o reported by the QA'd radiologist and the pathology grader reported by the QA'ing radiologist, the presence of a discrepancy y can be determined. The five different discrepancy values can be represented as:

y∈{Agree,1-degree undercall,1-degree overcall,2-degree undercall,2-degre overcall} Eq. (2)

The discrepancy value y can be more specifically defined in terms of o∈G (e.g., the grade given by the QA'd radiologist) and s∈G (e.g., the true underlying grade) as follows:

$$f ( o , r ) = \{ \begin{matrix}Agree & if o = s \\ 1 - degree undercall & if o + 1 = s \\ 1 - degree overcall & if 0 - 1 = s \\ 2 - degree undercall & if 0 + 1 < s \\ 2 - degree overcall & if o - 1 > s\end{matrix} . \qquad Eq . ( 3 )$$

That is, the discrepancy value defined in Eq. (3) captures the discrepancy of o with respect to r (e.g., the discrepancy of the QA'd radiologist's grade with respect to the QA'ing radiologist's grade). Note that the grade r given by the QA'ing radiologist is used as a proxy for the true underlying grade s, because in practice the true underlying grade s is not available. It is further noted that alternative discrepancy mappings and/or nomenclatures can be utilized to characterizer severity grades, discrepancies, and/or diagnostic errors, all without departing from the scope of the present disclosure.

EDPM generation begins with a Bayesian approach to determining the probability of the patient's true pathology grade sin a QA'd exam, given the pathology grade o reported by the QA'd radiologist, and the pathology grade r assessed by the QA'ing radiologist. In other words, the QA'ing radiologists' error detection probability can be determined based on the conditional probability of s given o and r:

$$\begin{matrix}p ( s \vert o , r ) = \frac{p ( o , r \vert s ) p ( s )}{p ( o , r )} \\ = \frac{p ( o , r \vert s ) p ( s )}{{\sum }_{{s}^{′} ∈ 𝒢} p ( o , r \vert {s}^{′} ) p ( {s}^{′} )} \\ = \frac{p ( o \vert s ) p ( r \vert s ) p ( s )}{{\sum }_{{s}^{′} ∈ 𝒢} p ( o \vert {s}^{′} ) p ( r \vert {s}^{′} ) p ( {s}^{′} )} \\ = \frac{{p}_{s , o}^{{QA}^{′} d} {p}_{s , r}^{{QA}^{′} ing} {p}_{s}^{s}}{{\sum }_{{s}^{′} ∈ 𝒢} {p}_{{s}^{′}}^{{QA}^{′} d} {p}_{{s}^{′} , r}^{{QA}^{′} ing} {p}_{{s}^{′}}^{s}}\end{matrix} \qquad Eq . ( 4 )$$

Here, p^{QA'd} and p^{QA'ing} are conditional probability distributions, estimated from the initial set of QA'd exams for which QA'ing radiologists have provided multiple secondary reads. More particularly:
p^{QA'd} is the conditional probability that the QA'd radiologist will report a finding of a specific grade, given that the QA'd exam contains a pathology with a true grade of s;
p^{QA'ing} is the conditional probability that the QA'ing radiologist will report a finding of a specific grade, given that the QA'd exam contains a pathology with a true grade of s; and
p^{s} is the underlying, observed, or historical probability that the QA'd exam contains a pathology with a true grade of s.

In general, the conditional probabilities p(s|o,r) can be understood to quantify a belief in the grade levels r provided by the QA'ing radiologists. While this information alone provides valuable insight, this conditional probability expression can be further refined by extending it as p((s,ƒ(o,s))|(r,ƒ(o,r))), which quantifies a belief in both the grade levels r and the discrepancy values/diagnostic errors ƒ(o,r) that are provided by the QA'ing radiologists. Note that the EDPM can be seen as representing these conditional probabilities p((s,ƒ(o,s))|(r,ƒ(o,r))) calculated for the initial set of diagnostic imaging exams with multiple independent QA reviews.

However, Equation (4) is not simply solved for these conditional probabilities. Notably, the various p^{QA'd}, p^{QA'ing} and p^{s} values are not known; nor are they easily determined, recalling that conventional approaches are able to achieve only limited insight into these values through the use of gold standard follow-up tests. As such, conventional QA approaches and solutions are at best able to make limited characterizations of p^{QA'd}, p^{QA'ing} and p^{s} values after the fact, not during or as a part of the QA process itself. For example, at the completion of a conventional QA review, multiple gold standard follow-up tests would have to be ordered and performed for the QA'd exams, and the test results would then have to be analyzed against the initial QA review—in effect, a separate and distinct QA review of the initial QA review. Not only would such an approach be logistically challenging and cost prohibitive (e.g., from having to schedule follow-up gold standard tests with each patient from the QA'd exams), but it would also be cumbersome and slow, facing substantial difficulties in updating to reflect changes in the composition of the group of radiologists, let alone increases and decreases in performance level/diagnostic accuracy.

Accordingly, the presently disclosed BIRAR-based approach seeks to solve these challenges and more, by not only determining diagnostic error rates and incorporating them into a single, streamlined QA process, but also further providing for efficient mechanisms to both update/refine diagnostic error rate measurements and scale the measurements as broadly or narrowly as desired (e.g., to individual radiologists, various groupings of radiologists, or a radiologist population as a whole). To achieve this functionality, the BIRAR-based approach provides systems and methods for flexibly and efficiently determining the previously unknown p^{QA'd}, p^{QA'ing} and p^{s} values.

For example, the p^{QA'd}, p^{QA'ing} and p^{s} values can be inferred, beginning from a set of observed data D_{EDPM} that is obtained from the initial quantity of QA'd exams that receive multiple secondary reads from QA'ing radiologists, where:

D_{EDPM}={(o₁,(r_{1,1}. . . r_{1,N}_{_{reviews}})), . . . ,(o_{N}_{_{studies}},(r_{N}_{_{studies}}_{,1}, . . . ,r_{N}_{_{studies}}_{,N}_{_{reviews}}))} Eq. (5)

where N_{studies} is the number of QA'd exams in the initialization set and N_{reviews} is the number of independent secondary reads performed for each exam in the initialization set. o₁ is the QA'd radiologist read of the first exam in the initialization set; o_{N}_{_{studies}} is the QA'd radiologist read of the N_{studies}^{th} exam. Similarly, r_{1,1} is the first QA'ing radiologist read of the first exam in the initialization set; r_{1,N}_{_{reviews}} is the N_{reviews}^{th} QA'ing radiologist read of the first exam in the initialization set; and r_{N}_{_{studies}}_{,N}_{_{reviews}} is the N_{reviews}^{th} QA'ing radiologist read of the N_{studies}^{th} exam. More generally, for each exam of the N_{studies} QA'd exams in the initialization set, the set of observed data D_{EDPM} contains a single QA'd radiologist read o and N_{reviews} different QA'ing radiologist reads.

The estimation proceeds on the basis of the following hierarchical generative model for the discrepancies y (a Stan implementation of the model is detailed in Appendix A):

p^{s}˜Dirichlet(1),

pₖ^{QA'd}˜Dirichlet(1),k=0,1, . . . ,N_{grades},

pₖ^{QA'ing}˜Dirichlet(1),k=0,1, . . . ,N_{grades},

sᵢ|p^{s}˜Categorical(p^{s}),i=1,2, . . . ,N_{studies},

oᵢ|pₛ_{ᵢ}^{QA'd}˜Categorical(pₛ_{ᵢ}^{QA'd}),i=1,2, . . . ,N_{studies},

r_{i,j}|pₛ_{ᵢ}^{QA'ing}˜Categorical(pₛ_{ᵢ}^{QA'ing}),i=1,2, . . . ,N_{studies},j=1,2 . . . ,N_{reviewers},

y_{i,j}=ƒ(oᵢ,r_{i,j}),i=1,2, . . . ,N_{studies},j=1,2, . . . ,N_{reviewers}. Eq. (6)

The three probabilities of interest—p^{s}, pₖ^{QA'd}, and pₖ^{QA'ing}—are modeled as Dirichlet distributions. In the example above, these Dirichlet distributions are shown as being parameterized with α=1, given that hierarchical priors for pₖ^{QA'd} and pₖ^{QA'ing} are not known or otherwise available. In some embodiments, it is possible to estimate one or more hierarchical priors for the Dirichlet distributions (see, for example, the ‘Performance & Results’ section, in which estimated priors α^{QA'd} and α^{AQ'ing} are used to calibrate probability distributions used as input for a simulated BIRAR-based process).

The three Dirichlet distributions are provided as priors to the corresponding categorical distributions sᵢ, oᵢ, and r_{i,j}. Note that because the Dirichlet distribution is the conjugate prior distribution of the categorical distribution, the model design seen in Eq. (6) advantageously permits the iterative incorporation of new observations and the updating of knowledge relating to the diagnostic error rates/error detection probabilities that will be used in generating the EDPM.

In terms of the EDPM that will ultimately be generated, note that in Eq. (6), pₖ^{QA'ing} is shared across QA'ing radiologists, meaning that a single EDPM will be generated. However, it is also contemplated that EDPMs could be generated for each QA'ing radiologist separately and/or estimated for sub-sets/groups of QA'ing radiologists by adjusting the manner in which pₖ^{QA'ing} is configured (e.g., by calculating multiple instances of pₖ^{QA'ing} with the same granularity that is desired in the output mix of EDPMs).

The posterior probability density function of Eq. (6) is proportional to the joint probability density function:

$$p ( θ \vert {𝒟}_{EDPM} ) ∝ p ( {𝒟}_{EDPM} , θ ) = p ( {𝒟}_{EDPM} \vert θ ) p ( θ ) = \\ {\prod }_{i = 1}^{{N}_{studies}} [ {\sum }_{s ∈ 𝒢} [ Categorical ( s \vert {p}^{s} ) Categorical ( {o}_{i} \vert {p}_{s}^{{QA}^{′} d} ) {\prod }_{j = 1}^{{N}_{reviews}} Categorical ( {r}_{i , j} \vert {p}_{s}^{{QA}^{′} ing} ) ] ] \\ [ {\prod }_{s ∈ 𝒢} Dirichlet ( {p}_{s}^{{QA}^{′} d} \vert 1 ) Dirichlet ( {p}_{s}^{{QA}^{′} ing} \vert 1 ) ] Dirichlet ( {p}^{s} \vert 1 ) , \qquad Eq . ( 7 )$$

where D_{EDPM} is the same observed data set described in Eq. (5). Additionally, it is noted that in the formulation given in Eq. (7), the discrete variables sᵢ=1, 2, . . . , N_{studies} are marginalized out.

Using the model of Eq. (6) (e.g., see also Appendix A, detailing a Stan implementation of the model of Eq. (6)), samples can be produced from the density p(θ|D_{EDPM}). For example, in some embodiments the model can be run with four chains with 500 warm-up iterations and 500 sampling iterations per chain, although it is appreciated that various other configurations can be utilized without departing from the scope of the present disclosure. R-hat and HMC-NUTS (Hamiltonian Monte Carlo and No-U-Turn-Sampler) specific diagnostics can be used to minimize or eliminate sampling issues.

In particular, samples p^{s}^{⁽ⁱ⁾}, pₖ^{QA'd}^{⁽ⁱ⁾}, pₖ^{QA'ing}^{⁽ⁱ⁾} are obtained for k=1, 2, . . . , N_{grades} and i=1, 2, . . . , Nₛₐₘₚₗₑₛ. Using these posterior samples, additional posterior samples can be produced for p(s|o,r)⁽ⁱ⁾ and p((s,ƒ(o,s))|(r,ƒ(o,r)))⁽ⁱ⁾, where i=1, 2, . . . , Nₛₐₘₚₗₑₛ—recalling that these two conditional probabilities can be used to drive the generation of one or more EDPMs (such as the example EDPM shown in FIG. 1). In some embodiments, the BIRAR-based approach can estimate and use [symbol] [p(s|o, r)|D_{EDPM}] and [symbol] [p((s,ƒ(o,s))|(r,ƒ(o,r)))|D_{EDPM}] from the posterior samples, and thereby generate the desired EDPM(s).

### Estimation of Error Rates

Error rate estimation aims to quantify the rate of diagnostic errors made by QA'd radiologists (e.g., by using the EDPM(s) and BIRAR-based approach initialized by the set of multiple independent QA'd exams as described above). Notably, once the EDPM 100 is estimated, future QA'd exams need only be subjected to a single QA review. Instead of using the results of the QA reviews to directly calculate diagnostic error rates of the QA'd radiologist (e.g., treating each discrepancy as evidence of a diagnostic error, as in many conventional approaches), the generated EDPM is instead used to transform the results of each single QA review into a probability distribution over the five possible diagnostic error categorizations (e.g., No Error/Agree, 1-Degree Undercall, 1-Degree Overcall, 2-Degree Undercall, 2-Degree Overcall). These transformed probability distributions can be aggregated across all of the QA'd exams corresponding to a given QA'd radiologist, such that an expected value can be calculated for the given QA'd radiologist's overall rate of diagnostic error (and/or the rate at which diagnostic errors of specific types occur, e.g., the rate of 2-Degree Undercalls or 1-Degree Overcalls).

With respect to diagnostic error rate estimation for QA'd radiologists (e.g., BIRAR-based QA), consider the following example. It might be desirable to know the probability that a given QA'd radiologist makes a 1-degree overcall error when the true pathology severity grade in the QA'd exam is Grade 0 (e.g., a 1-degree overcall error indicates that the QA'd radiologist indicated Grade 1). However, the estimation of the rate of diagnostic error is not a trivial problem, because the underlying severity grade s is never observed directly, as the presently disclosed BIRAR-based approach does not require or make use of gold standard follow-up tests. Described below are three different approaches for estimating diagnostic error rates.

1. Estimating Error Rates without a Correction for Imperfect QA'Ing Radiologists

This approach assumes that the QA'ing radiologists do not make diagnostic errors of their own (an assumption that, in practice, is not valid). The severity grades r given by the QA'ing radiologists are treated as a perfect proxy for the underlying true severity grade s (e.g., r=s).

Let N_{reviews}=1 for all i=1, 2, . . . , N_{studies}. Moreover, consider the following QA data set:

[symbol]

={(r₁,ƒ(o₁,r₁)),(r₂,ƒ(o₂,r₂)), . . . ,(r_{N}_{_{studies}},ƒ(o_{N}_{_{studies}},r_{N}_{_{studies}}))} Eq. (8)

Note that N_{reviews}=1 for each of the N_{studies} because, once initialized with the EDPM, only a single QA'ing radiologist is required to provide a secondary read for the presently disclosed BIRAR-based QA process to function. Furthermore, it is not necessary for the QA'ing radiologists providing these single QA reviews to have been included in the selection of QA'ing radiologists used to initialize the EDPM by providing the series of multiple independent reviews collected for single QA'd exams. Nor is it necessary for the QA'ing radiologist to have a higher level of skill/lower error rate than either the radiologist being subjected to QA review, or a higher level of skill/lower error rate than the QA'ing radiologists whose secondary reviews were used in generating the EDPM.

Therefore, the set D comprises a severity grade and discrepancy value from a single QA'ing radiologist's secondary read. The maximum likelihood estimates (MLE) of the rates of discrepancies under the categorical model without correcting for imperfect QA'ing radiologists and given a severity grade g∈G are given as follows:

$${\hat{p}}_{MLE} ( Agree \vert g ) = \frac{1}{{\sum }_{i = 1}^{{N}_{studies}} [ {r}_{i} = g ]} {\sum }_{i = 1}^{{N}_{studies}} [ f ( {o}_{i} , {r}_{i} ) = Agree ∧ {r}_{i} = g ] , \\ {\hat{p}}_{MLE} ( 1 - degree undercall \vert g ) = \frac{1}{{\sum }_{i = 1}^{{N}_{studies}} [ {r}_{i} = g ]} {\sum }_{i = 1}^{{N}_{studies}} [ f ( {o}_{i} , {r}_{i} ) = 1 - degree undercall ∧ {r}_{i} = g ] , \\ {\hat{p}}_{MLE} ( 1 - degree overcall \vert g ) = \frac{1}{{\sum }_{i = 1}^{{N}_{studies}} [ {r}_{i} = g ]} {\sum }_{i = 1}^{{N}_{studies}} [ f ( {o}_{i} , {r}_{i} ) = 1 - degree overcall ∧ {r}_{i} = g ] , \\ {\hat{p}}_{MLE} ( 2 - degree undercall \vert g ) = \frac{1}{{\sum }_{i = 1}^{{N}_{studies}} [ {r}_{i} = g ]} {\sum }_{i = 1}^{{N}_{studies}} [ f ( {o}_{i} , {r}_{i} ) = 2 - degree undercall ∧ {r}_{i} = g ] , \\ {\hat{p}}_{MLE} ( 2 - degree overcall \vert g ) = \frac{1}{{\sum }_{i = 1}^{{N}_{studies}} [ {r}_{i} = g ]} {\sum }_{i = 1}^{{N}_{studies}} [ f ( {o}_{i} , {r}_{i} ) = 2 - degree overcall ∧ {r}_{i} = g ] , \qquad Eq . ( 9 )$$

$$where$$

$$[ x ] = \{ \begin{matrix}1 & if x is true \\ 0 & otherwise\end{matrix} . \qquad Eq . ( 10 )$$

As noted previously, the approach above assumes that the severity grades given by QA'ing radiologists are a perfect proxy for the underlying severity grades. If the proxy is not perfect, as it is not in practice, then the estimated error rates in Eq. (9) may be biased.

2. Estimating Error Rates with a Correction for Imperfect QA'Ing Radiologists

In some embodiments, the biased estimated error rates given by Eq. (9) can be addressed by implementing a correction for the imperfect nature of the secondary reads provided by QA'ing radiologists. Let N_{reviews}=1 for all i=1, 2, . . . , N_{studies}. Moreover, consider the following QA data set:

[symbol]

={(r₁,ƒ(o₁,r₁)),(r₂,ƒ(o₂,r₂)), . . . ,(r_{N}_{_{studies}},ƒ(o_{N}_{_{studies}},r_{N}_{_{studies}}))},

which is the same as the data set given in Eq. (8).

With the correction for imperfect QA'ing radiologists, the maximum likelihood estimates (MLE) from Eq. (9) become:

$${\hat{p}}_{MLE} ( Agree \vert g ) = \frac{1}{{\sum }_{i = 1}^{{N}_{studies}} p ( g \vert ( {r}_{i} , f ( {o}_{i} , {r}_{i} ) ) )} {\sum }_{i = 1}^{{N}_{studies}} p ( ( g , Agree ) \vert ( {r}_{i} , f ( {o}_{i} , {r}_{i} ) ) ) , \\ {\hat{p}}_{MLE} ( 1 - degree undercall \vert g ) = \frac{1}{{\sum }_{i = 1}^{{N}_{studies}} p ( g \vert ( {r}_{i} , f ( {o}_{i} , {r}_{i} ) ) )} {\sum }_{i = 1}^{{N}_{studies}} p ( ( g , 1 - degree undercall ) \vert ( {r}_{i} , f ( {o}_{i} , {r}_{i} ) ) ) , \\ {\hat{p}}_{MLE} ( 1 - degree overcall \vert g ) = \frac{1}{{\sum }_{i = 1}^{{N}_{studies}} p ( g \vert ( {r}_{i} , f ( {o}_{i} , {r}_{i} ) ) )} {\sum }_{i = 1}^{{N}_{studies}} p ( ( g , 1 - degree overcall ) \vert ( {r}_{i} , f ( {o}_{i} , {r}_{i} ) ) ) , \\ {\hat{p}}_{MLE} ( 2 - degree undercall \vert g ) = \frac{1}{{\sum }_{i = 1}^{{N}_{studies}} p ( g \vert ( {r}_{i} , f ( {o}_{i} , {r}_{i} ) ) )} {\sum }_{i = 1}^{{N}_{studies}} p ( ( g , 2 - degree undercall ) \vert ( {r}_{i} , f ( {o}_{i} , {r}_{i} ) ) ) , \\ {\hat{p}}_{MLE} ( 2 - degree overcall \vert g ) = \frac{1}{{\sum }_{i = 1}^{{N}_{studies}} p ( g \vert ( {r}_{i} , f ( {o}_{i} , {r}_{i} ) ) )} {\sum }_{i = 1}^{{N}_{studies}} p ( ( g , 2 - degree overcall ) \vert ( {r}_{i} , f ( {o}_{i} , {r}_{i} ) ) ) , \qquad Eq . ( 11 )$$

$$where$$

$$p ( g \vert ( {r}_{i} , f ( {o}_{i} , {r}_{i} ) ) ) = p ( ( g , Agree ) \vert ( {r}_{i} , f ( {o}_{i} , {r}_{i} ) ) ) + p ( ( g , 1 - degree undercall ) \vert ( {r}_{i} , f ( {o}_{i} , {r}_{i} ) ) ) + p ( ( g , 1 - degree overcall ) \vert ( {r}_{i} , f ( {o}_{i} , {r}_{i} ) ) ) + p ( ( g , 2 - degree undercall ) \vert ( {r}_{i} , f ( {o}_{i} , {r}_{i} ) ) ) + p ( ( g , 2 - degree overcall ) \vert ( {r}_{i} , f ( {o}_{i} , {r}_{i} ) ) ) . \qquad ( 12 )$$

Instead of using the QA'ing radiologist severity grades, r, as a direct proxy for the underlying severity grade (e.g., as was done in Eq. (9)), the error estimation approach described in Eq. (11) instead uses the conditional probabilities p((s,ƒ(o,s)|(r,ƒ(o,r)). That is, the hard labels r,(ƒ(o,r)) are translated into the soft labels p((s,ƒ(o,s)|(r,ƒ(o,r)). Note that the error estimation with imperfect QA'ing radiologist correction contains an implicit assumption, which is that the data set D (with a single secondary reviews per QA'd exam) and the data set D_{EDPM} (with multiple independent secondary reviews per QA'd exam; used to generate the EDPM) have similar data-generating distributions. If this assumption does not hold, then the correction approach described in Eq. (11) becomes biased as well. In such a situation, it may be necessary or desirable to re-calibrate the EDPM, either by collecting a supplemental set of QA'd exams having multiple independent secondary reviews and then updating the existing EDPM, or by collecting a new set of QA'd exams having multiple independent secondary reviews and then generating an entirely new EDPM. The motivation behind such a process might be that a divergence between the data-generating distributions of D and D_{EDPM} is driven by some type of change in the composition, skill level, performance, etc., between the QA'ing radiologists who were at some point in the past sampled to obtain D_{EDPM}, and the QA'ing radiologists who are currently participating in the BIRAR-based QA process and providing the reads contained in D. In other words, a divergence between the data-generating distributions for D and D_{EDPM} likely indicates that the current population of radiologists providing the single QA reviews has drifted from the earlier population of radiologists who provided the multiple independent QA reviews that initialized D_{EDPM} and the EDPM, and consequently, a recalibration may be needed.

In some embodiments, the BIRAR-based QA process can automatically monitor its performance, using for example, one or more of the metrics described in the Performance & Results section for quantifying the performance of a simulated BIRAR-based QA. In response to determining that the data sets D and D_{EDPM} may have drifted, the BIRAR-based QA can perform one or more of the following: generate automatic warnings or messages to administrators or other users; automatically select certain imaging exams and groups of QA'ing radiologists and then schedule the groups to provide multiple independent secondary reads of the selected imaging exams by the groups; compile the results of the additional multiple reviewed imaging exams into a D_{EDPM_Update} data set and then generate one or more updated EDPMs; generate updated EDPMs in substantially real-time as the results of the additional multiple reviewed imaging exams become available (e.g., without waiting to compile them into D_{EDPM_Update}); etc.

3. Estimating Error Rates Based on QA'Ing Radiologist Majority

A third approach for estimating diagnostic error rates can be based on a majority or consensus approach. Consider the following data set:

$$𝒟 = \{ \{ ( {r}_{1 , 1} , f ( {o}_{1} , {r}_{1 , 1} ) ) , … , ( {r}_{1} {N}_{reviews} , f ( {o}_{1} , {r}_{1} , {N}_{reviews} ) ) \} , \\ \{ ( {r}_{2 , 1} , f ( {o}_{2} , {r}_{2 , 1} ) ) , … , ( {r}_{2} , {N}_{reviews} , f ( {o}_{2} , {r}_{2} , {N}_{reviews} ) ) \} , \\ ⋮ \\ \{ ( {r}_{{N}_{studies} , 1} , f ( {o}_{{N}_{studies}} , {r}_{{N}_{studies} , 1} ) ) , … , ( {r}_{{N}_{studies} , {N}_{reviews} , f ( {o}_{{N}_{studies}} , {r}_{{N}_{studies} {N}_{reviews}} ) ) \} \} .} \qquad Eq . ( 13 )$$

In this data set, each QA exam is associated with N_{reviews} provided as secondary reads by QA'ing radiologists. From this data set having N_{reviews} per exam, a majority agreement data set can be derived by filtering out the exams without majority agreement, such that:

$${𝒟}_{majority} = \{ ( y , z ) \vert x ∈ 𝒟 ∧ ( y , z ) ∈ x ∧ {\sum }_{{x}^{′} ∈ x} [ ( y , x ) = {x}^{′} ] > \frac{{N}_{reviews}}{2} \} \qquad Eq . ( 14 )$$

More particularly, this approach generates D_{majority} by keeping only those exams in which there are more than N_{reviews}/2 QA'ing radiologists that agree with one another. Additionally, this single majority opinion shared between the QA'ing radiologists can be used as a single assessment for the given QA'd exam: |D_{majority}|≤|D| due to the filtering of Eq. (14), and as such, only a single QA'ing radiologist read r is needed per exam in D_{majority} instead of having N_{reviews} QA'ing radiologist reads per exam.

After filtering out the studies without majority agreement, the error estimation without imperfect QA'ing radiologist correct approach can be applied as described in Eq. (9), achieving greater accuracy without the greater computational overhead associated with the error estimation approach described in Eq. (11), which does perform additional steps for imperfect QA'ing radiologist correction. Motivating the majority approach error estimation described above with respect to Eq. (14) is an understanding that the majority opinion in D_{majority} is a better proxy of the underlying pathology severity grade than a single QA'ing radiologist opinion is.

### BIRAR: Numerical Example

With reference to Equation (4)'s formulation of p (s|o,r), provided below is a numerical example demonstrating an application of the presently disclosed BIRAR-based approach to determining QA'ing radiologists' error detection probability.

For instance, consider the following example with the following starting values (which can be obtained via the sampling process described in Eq. (6) and Eq. (7), in which samples p^{s}^{⁽ⁱ⁾}, pₖ^{QA'd}^{⁽ⁱ⁾}, pₖ^{QA'ing}^{⁽ⁱ⁾} are obtained for k=1, 2, . . . , N_{grades} and i=1, 2, . . . , Nₛₐₘₚₗₑₛ:

N_{grades}=3,p^{s}=(0.5 0.3 0.2)^{T},

p₀^{QA'd}=(0.85 0.1 0.05)^{T},p₁^{QA'd}=(0.2 0.6 0.2)^{T},p₂^{QA'd}=(0.1 0.25 0.65)^{T},

p₀^{QA'ing}=(0.9 0.075 0.025)^{T},p₁^{QA'ing}=(0.15 0.7 0.15)^{T},p₂^{QA'ing}=(0.1 0.2 0.7)^{T}, Eq. (15)

The values of p₀^{QA'd} indicate that when the pathology has a true Grade 0, the QA'd radiologist will assign Grade 0 85% of the time, Grade 1 10% of the time, and Grade 2 5% of the time (e.g., a total error rate of 15% for Grade 0 pathologies). p₁^{QA'd} and p₂^{QA'd} reflect the same for when the pathology has a true Grade 1 and 2, respectively.

The values of p₀^{QA'ing} indicate that when the pathology has a true Grade 0, the QA'ing radiologist will assign Grade 0 90% of the time, Grade 1 7.5% of the time, and Grade 2 2.5% of the time (a total error rate of 9% for Grade 0 pathologies). p₁^{QA'ing} and p₂^{QA'ing} reflect the same for when the pathology has a true Grade 1 and 2, respectively.

Given the above values, then the following conditional probabilities can be calculated (recalling that s is the true pathology grade, o is the QA'd radiologist grade, and r is the QA'ing radiologist grade):

$$p ( s \vert o , r ) \approx \begin{matrix}\begin{matrix}s = 0 & s = 1 & s = 2\end{matrix} \\ ( \begin{matrix}0.972 & 0.023 & 0.005 \\ 0.409 & 0.539 & 0.051 \\ 0.316 & 0.268 & 0.416 \\ 0.584 & 0.351 & 0.065 \\ 0.027 & 0.902 & 0.072 \\ 0.02 & 0.427 & 0.553 \\ 0.506 & 0.601 & 0.292 \\ 0.027 & 0.601 & 0.372 \\ 0.006 & 0.089 & 0.904\end{matrix} )\end{matrix} \begin{matrix}o = 0 , & r = 0 \\ o = 0 , & r = 1 \\ o = 0 , & r = 2 \\ o = 1 , & r = 0 \\ o = 1 , & r = 1 \\ o = 1 , & r = 2 \\ o = 2 , & r = 0 \\ o = 2 , & r = 1 \\ o = 2 , & r = 2\end{matrix} \qquad Eq . ( 16 )$$

Using a similar approach as in Equation (4), the conditional probabilities p((s,ƒ(o,s))|(r,ƒ(o,r))) can be calculated. These probabilities quantify a belief in the grade levels and discrepancy values (r,ƒ(o,r)) given by the QA'ing radiologists.

Still using the same example values given in (15), consider the specific case of o=0 and r=1, which results in ƒ(o,r)=1-degree undercall discrepancy identified by the QA'ing radiologist. Accordingly, the probabilities p((s,ƒ(o,s))|(r,ƒ(o,r))) are then:

p((0,Agree)|(1,1-degree undercall))≅0.409

p((1,1-degree undercall)|(1,1-degree undercall))≅0.539

p((2,2-degree undercall)|(1,1-degree undercall))≅0.051. Eq. (17)

Note that both 1-degree overcall and 2-degree overcall are impossible if o=0 (likewise, 1-degree undercall and 2-degree undercall are impossible if o=2; etc.) Additionally, if the discrepancy function ƒ(x,·) for some x is a many-to-one function, e.g.:

∃x∈ [symbol] ∃y,z∈ [symbol] (ƒ(x,y)=ƒ(x,z)∧y≠z), Eq. (18)

then in some embodiments it can be desirable to aggregate over those equivalence classes, as a focus of the BIRAR-based approach is on the pairs made of a severity grade and a discrepancy value.

The conditional probability values determined in Eq. (4) are thus the probability values used to populate the EDPM, as the EDPM is generated to characterize the conditional probabilities that a diagnostic error of a specific type is present in a QA'd exam, given the presence of a specific discrepancy flagged by a QA'ing radiologist who performed a secondary read of the QA'd exam.

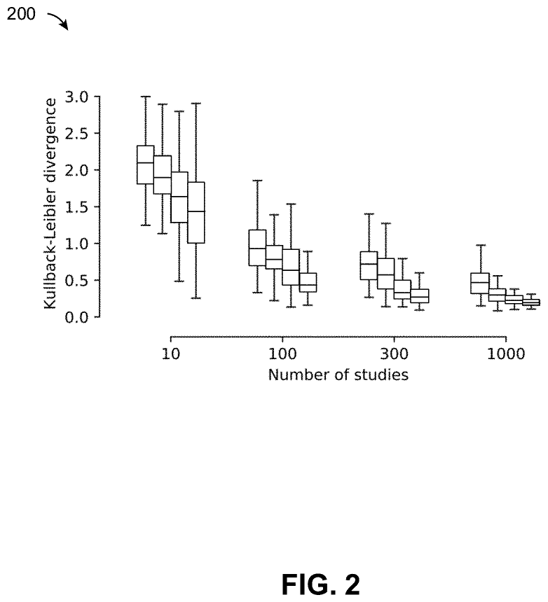

Consider again the same scenario outlined with respect to the example values given in Eq. (15). Using simulated data (e.g., see below section discussion performance & results with respect to a BIRAR simulation), the Kullback-Leibler divergence of the true distribution from the estimated distribution can be studied as a function of the number of studies and the number of reviews. The results of the Kullback-Liebler divergence (KLD) are illustrated in the graph 200 of FIG. 2. In particular, graph 200 is a boxplot indicating a series of results that were estimated based on 100 simulations. Within each of the four clusters of boxes (e.g., a total of four clusters, each having four boxes, are shown in graph 200), the leftmost box is obtained for number of reviews=2; the second from the left box is obtained for number of reviews=3; the third from the left box is obtained for number of reviews=5; and the rightmost box is obtained for number of reviews=10.

### Performance & Results: Forward Simulation of BIRAR QA Process

### Summary and Study Design

A simulation study was used to evaluate the performance of BIRAR and four other methods for measuring diagnostic error rates. The results of the simulation study show that the presently disclosed BIRAR (Bayesian Inter-Reviewer Agreement Rate) approach for measuring diagnostic error rates is more accurate and requires fewer secondary assessments compared to alternate approaches that use three independent secondary assessments of each QA'd study and where majority agreements about discrepancies are treated as evidence of a diagnostic error. Additionally, the presently disclosed BIRAR approach is robust with respect to the ability to accurately measure diagnostic error rates even when the secondary QA assessments are performed by radiologists who themselves have higher error rates than the radiologists covered by the QA program.

The simulation study used a generative model to simulate:
A set of diagnostic imaging exams performed by a set of QA'd radiologists that had predefined probabilities for making diagnostic errors of various types. (QA'd radiologists are those radiologists whose original reads are subject to the QA review process); and
Results of secondary QA reviews of imaging exams performed by QA'ing radiologists who are also assigned predefined probabilities for making interpretive errors. (QA'ing radiologists are those radiologists who provide secondary reads as part of the QA review process).

Five approaches for measuring the diagnostic error rates of the QA'd radiologists were then evaluated using the results of the simulated QA reviews performed by the QA'ing radiologists. Since this is a simulation study, an explicit comparison can be made between the actual rate of diagnostic errors in the QA'd exams and the diagnostic error rate calculated by each of the measurement approaches based on the use of secondary QA reviews. The five methods for calculating diagnostic error rates that were evaluated in the study are listed below:
1. The presently disclosed novel “Bayesian Inter-Reviewer Agreement Rate” (BIRAR) approach, where an initial set of QA'd exams are subjected to three independent QA reviews by QA'ing radiologists in order to characterize the error rates among the QA'ing radiologists, and the results of this initial analysis are then used together with the data generated from an additional set of QA'd studies subjected to single QA reviews by QA'ing radiologists to calculate the diagnostic error rates of the QA'd radiologists.
2. The “Perfect QA'ing Radiologists” approach, in which the QA'ing radiologists always correctly detect and grade pathology, so that any discrepancy flagged in a secondary QA review of an exam is evidence of a diagnostic error in the exam. This approach is clearly not practical in reality since QA'ing radiologists are not infallible, but the “Perfect QA Reviewing Radiologists” scenario is included in this simulation study to illustrate the amount of statistical uncertainty that would be present in an error rate measurement performed by “perfect” QA'ing radiologists due to the evaluation of a finite sample of QA'd Exams.
3. The “Single Review” approach, in which a single QA review is performed by one of the QA'ing radiologists for each of the QA'd Exams and the results of the QA reviews are treated as though the QA'ing radiologists are perfect (e.g., as in the “Perfect QA Reviewing Radiologists” approach), even though the QA'ing radiologists have non-zero probabilities of making errors. This method is similar to the approach taken in many peer review processes that are incorporated into radiology QA programs. Comparing the performance of this approach with the BIRAR approach shows how well the BIRAR approach is able to correct for the QA'ing radiologists' errors.
4. The “Majority Panel” approach, in which each QA'd Exam is independently reviewed three times by QA'ing radiologists, and the majority of the QA'ing radiologists have to flag the same discrepancy for a diagnostic error to be considered present in the QA'd exam. All of these first four methods in this list are simulated in a manner that uses a fixed and consistent number of total secondary QA reviews to assess the diagnostic error rates of the population of QA'd radiologists covered by the hypothetical QA program. This means that the Majority Panel approach, since it requires each QA'd Exam to be QA'd three times, is only able to assess one third as many exams per QA'd radiologist compared to the “Perfect QA'ing Radiologists” and “Single Review” approaches.
5. The “Majority Panel with 3× Total QA Review Volume” approach is the same as the “Majority Panel” approach described above, but the simulation uses three times as many aggregate QA exam reviews than the four methods listed above so roughly the same number of QA'd Exams are assessed per QA'd radiologist across all methods.

### Simulation of Imaging Exams and Calibration of Radiologist Error Rates

For the purposes of this simulation study, the hypothetical diagnostic imaging exams were defined to be ones in which a radiologist is tasked with the detection and ordinal grading of a single pathology type with three possible severity grades, which are enumerated as 0, 1, and 2. As illustrative examples, these simulated imaging exams could be considered to be modeling: knee MRI exams in which radiologists assess the ACL to be either normal, moderately injured (e.g. partially torn), or severely injured (e.g. completely torn); or lumbar spine MRI exams in which central canal stenosis at a specific motion segment is assessed to be not present or mild, moderate, or severe; etc.

The diagnostic error rates of the QA'd radiologist were determined by two sets of predefined parameters: (1) the probability that pathology severity grades 0, 1, and 2 will be diagnosed correctly or if the pathology will be misdiagnosed as one of the other grades, respectively; (2) the prevalence of imaging exams in which patients suffered from pathology grades 0, 1, and 2, respectively.

The probabilities that patient exams with true pathologies of specific grades would be diagnosed as the correct grade or incorrectly as one of the other grades, are modeled as Dirichlet distributions, which are calibrated using the following pattern of correct and incorrect diagnoses defined in the matrix α^{QA'd}:

TABLE 1
Example values of matrix α^{QA'd}

| | | QA'd Radiologist Grading | | |
|---|---|---|---|---|
| α^{QA'd} | | 0 | 1 | 2 |
| True Pathology | 0 | 80 | 4 | 2 |
| Grade in | 1 | 3 | 15 | 3 |
| Patient Exam | 2 | 2 | 4 | 20 |

Here, the first row in α^{QA'd} can be interpreted as, “out of 86 exams in which the true grading of the patient's pathology is ‘0’, the QA'd radiologist will correctly grade the exam as ‘0’ 80 times, incorrectly grade the exam as grade ‘1’ 4 times, and incorrectly grade the exam as “2” 2 times.” The second row in α^{QA'd} can be interpreted as, “out of 21 exams in which the true grading of the patient's pathology is ‘1’, the QA'd radiologist will correctly grade the exam as ‘1’ 15 times, incorrectly grade the exam as grade ‘0’ 3 times, and incorrectly grade the exam as “2” 3 times.” The third row in α^{QA'd} can be interpreted as, “out of 26 exams in which the true grading of the patient's pathology is ‘2’, the QA'd radiologist will correctly grade the exam as ‘2’ 20 times, incorrectly grade the exam as grade ‘0’ 2 times, and incorrectly grade the exam as “1” 4 times.”

The probabilities that QA'd Exams will have true pathology grades equal to 0, 1, and 2, respectively, were calibrated using the three probabilities defined in the vector p^{s}:

p^{s}=(0.5,0.3,0.2)

The vector p^{s} can be interpreted as “50% of the QA'd Exams have pathology of grade 0, 30% have pathology of grade 1, and 20% have pathology of grade 2.”

The rates of diagnostic errors defined for the QA'd radiologist in α^{QA'd} and the relative rates that QA'd Exams will have true pathology grades 0, 1, and 2, defined in p^{s}, were roughly informed by empirical data observed in active QA programs, but are effectively arbitrary choices that enable the relative performance of the five diagnostic error rate measurement methodologies to be compared to each other.

Given α^{QA'd} and p^{s} as defined above, the overall diagnostic error rate of the QA'd radiologists is 17% (e.g. in 83% of exams, the QA'd radiologist will grade the pathology in the QA'd Exam correctly). Further, the QA'd radiologists' rate of “two-degree errors”, which are defined to be errors where a grade 0 pathology is diagnosed as grade 2 or vice versa, is 3% (e.g. 97% of exams, the QA'd radiologist will grade the pathology in the exam with the patient's correct pathology grade or one degree off).

### Simulation of the QA Review Process and Calibration of QA'Ing Radiologists' Error Rates

The panel of QA'ing radiologists participating in the simulated QA program were defined to have three different profiles with respect to the probabilities that they would correctly detect and grade the pathology of interest in the secondary QA reviews. These probabilities were modeled in the same manner as the QA'd radiologists, described above, and were calibrated using the patterns of correct and incorrect diagnoses defined in the following three matrices α^{QA'ing,1}, α^{QA'ing,2}, and α^{QA'ing,3}, respectively:

TABLE 2
Example values of matrix α^{QA'ing, 1}

| | | QA'ing Radiologist Profile | | |
|---|---|---|---|---|
| | | 1: Diagnosed Grading | | |
| α^{QA'ing, 1} | | 0 | 1 | 2 |
| True Pathology | 0 | 100 | 4 | 2 |
| Grade in | 1 | 3 | 20 | 3 |
| Patient Exam | 2 | 2 | 4 | 25 |

TABLE 3
Example values of matrix α^{QA'ing, 2}

| | | QA'ing Radiologist Profile | | |
|---|---|---|---|---|
| | | 2: Diagnosed Grading | | |
| α^{QA'ing, 2} | | 0 | 1 | 2 |
| True Pathology | 0 | 80 | 4 | 2 |
| Grade in | 1 | 3 | 15 | 3 |
| Patient Exam | 2 | 2 | 4 | 20 |

TABLE 4
Example values of matrix α^{QA'ing, 3}

| | | QA'ing Radiologist Profile | | |
|---|---|---|---|---|
| | | 3: Diagnosed Grading | | |
| α^{QA'ing, 3} | | 0 | 1 | 2 |
| True Pathology | 0 | 60 | 4 | 2 |
| Grade in | 1 | 3 | 10 | 3 |
| Patient Exam | 2 | 2 | 4 | 15 |

Inspection of these three calibration matrices reveals that QA'ing Radiologist Profiles 1, 2 and 3, define lower, equal, and higher probabilities of errors, respectively, compared to what was defined for the QA'd radiologists above in matrix α^{QA'd}. Given p^{s} as also defined above, the diagnostic error rates of the QA'ing radiologists with Profile 1, defined by α^{QA'ing,1} (e.g., as depicted in Table 2), and Profile 3, defined by α^{QA'ing,3} (e.g., as depicted in Table 4), are 13% and 22%, respectively.

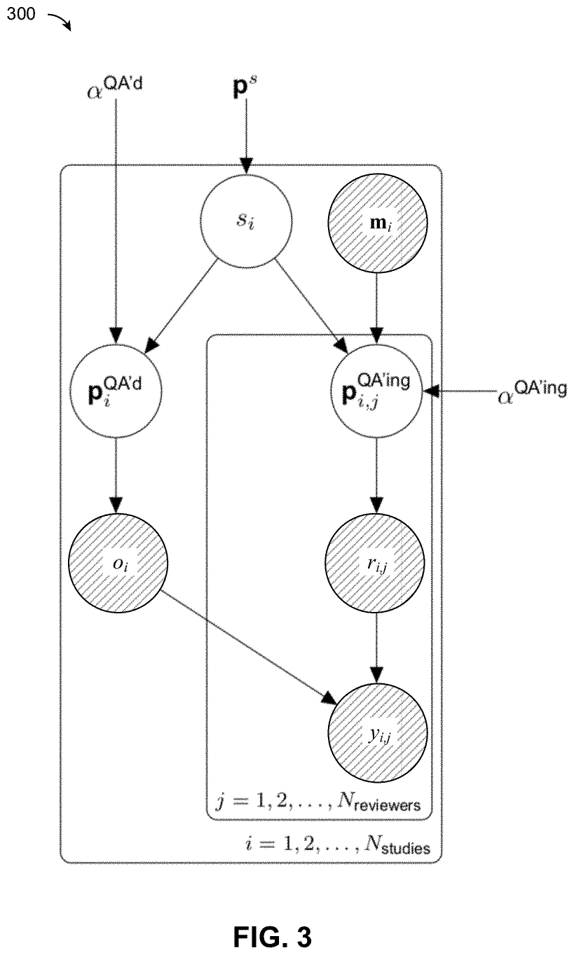

A schematic diagram 300 of an example of the presently disclosed QA review process is depicted in FIG. 3, which shows that the α^{QA'd} and α^{QA'ing} matrices, defined above, are used to calibrate the distributions of conditional probabilities, p^{QA'd} and p^{QA'ing} respectively, which are the probabilities that the QA'd and QA'ing radiologists will report a finding of a specific grade, given that the QA'd Exam contains pathology of a given severity grade, s. As described above, the vector p^{s} defines the probabilities that QA'd exams contain pathology of each severity grade. The variables o and r represent the pathology grade reported by the QA'd and QA'ing radiologist, respectively, and by comparing these two values the presence of a discrepancy, represented as y, can be determined. The subscripts i and j are indexes for the i^{th} QA'd Exam and the j^{th} secondary QA review of a given QA'd Exam. The variable m is a random permutation of integers between 1 and the number of QA'ing radiologists so that the simulation will randomly assign QA'ing radiologists to QA'd Exams.

### Example Simulation Parameters

Example simulation parameters used for each of the diagnostic error rate measurement methods evaluated in the study described above are depicted below in Table 5, including: the number of QA'd radiologists, QA'd Exams per QA'd radiologist, and QA reviews per QA'd Exam:

TABLE 5
Example simulation parameters used for each of
the diagnostic error rate measurement methods.

| | # QA Reviews | | | | |
|---|---|---|---|---|---|
| | Used for | | # of QA | # of QA'd | |
| Diagnostic Error | Inter-Reviewer | | Reviews | Studies | Total # |
| Rate Measurement | Agreement | # of QA'd | per QA'd | per QA'd | of QA |
| Method | Analysis | Radiologists | Exam | Radiologist | Reviews |
| BIRAR | 900 (300 | 100 | 1 | 81 | 9,000 |
| | Exams each | | | | |
| | QA'd 3x) | | | | |
| Perfect QA'ing | NA | 100 | 1 | 90 | 9,000 |
| Radiologists | | | | | |
| Single Review | NA | 100 | 1 | 90 | 9,000 |
| Majority Panel | NA | 100 | 3 | 30 | 9,000 |
| Majority Panel with | NA | 100 | 3 | 90 | 27,000 |
| 3x Total QA Review | | | | | |
| Volume | | | | | |

### Sensitivity Analyses

Two sensitivity analyses were included in this study to evaluate the impact that different assumptions about the QA'ing radiologists' error rates and different configurations of the BIRAR approach would have on the overall study results.

The first sensitivity analysis evaluated the impact of using a different number of QA'd Exams to estimate the EDPM. The simulation described above used the results of 300 QA'd Exams, each QA'd three times, to estimate the EDPM. In this sensitivity analysis, the simulation was re-run using the following alternate choices for the number of QA'd Exams used to estimate the EDPM: 30, 50, 100, 300, 900, 2700.

The second sensitivity analysis evaluated the impact of the QA'ing radiologists' error rates on the overall study results. In the simulation described above, the panel of QA'ing radiologists was modeled to include, in equal amounts, radiologists with error rates that were lower than, equal to, and greater than the QA'd radiologists. For this sensitivity analysis, the simulation was rerun with two alternate configurations. The first alternate configuration set all of the QA'ing Radiologists to have error rate profile 1, α^{QA'ing,1}, which as described above, defines a lower error rate than that of the QA'd radiologists. The second configuration set all of the QA'ing radiologists to have error rate profile 3, α^{QA'ing,3} which as described above, defines a higher error rate than that of the QA'd radiologists.

### Results

The initial step using BIRAR for diagnostic error rate measurement is the calculation of the EDPM. For example, the EDPM 100 of FIG. 1 can be generated as the resulting EDPM when using the simulation from 300 QA'd Exams that were each independently reviewed three times by QA'ing radiologists who themselves had error rates lower than, equal to, and higher than the QA'd radiologists, respectively. The matrix rows of the EDPM 100 indicate the “QA'ing Radiologist's Assessment” and the matrix columns of EDPM 100 indicate the correct assessment for the QA'd Exam. For example, the values in the fifth row indicate that if a QA'ing radiologist determines that the pathology in a QA'd Exam is Grade 1 and the QA'd radiologist committed a one-degree undercall error (e.g. the QA'd radiologist reported Grade 0), there is a 60% probability that the QA'ing radiologist is correct (shown in column five), a 34% probability that the QA'ing radiologist is mistaken and the QA'd radiologist is correct (shown in column one), and a 6% probability that they are both mistaken and the correct finding for the QA'd Exam is Grade 2 pathology. The probabilities in each row sum to one.

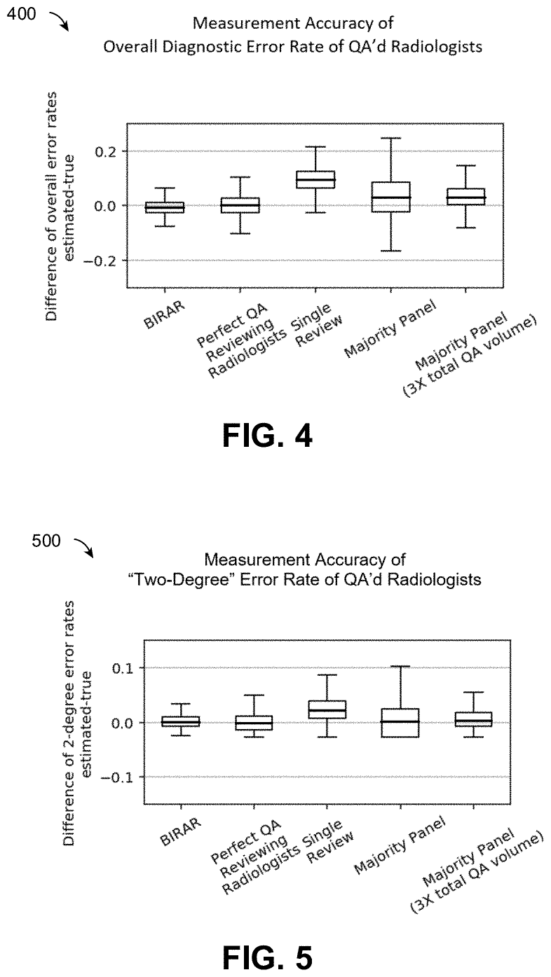

The results of the simulation of the five methods for measuring diagnostic error rates of a population of 100 QA'd radiologists, with the QA'ing radiologists comprised of an equal number of radiologists with lower, equal, and higher error rates than the QA'd radiologists, are depicted in the graph 400 of FIG. 4 and summarized below in Table 6. As described previously, each of the QA'd radiologists had an actual overall diagnostic error rate of 17%.

The graph 400 of FIG. 4 depicts boxplots of the difference between the measured and actual diagnostic error rates of 100 QA'd radiologists using the five error rate measurement methods simulated in this study. Standard boxplot notation is used, where the center line is the median value, the central box denotes 25^{th} to 75^{th} percentiles, and the whiskers extend to 25^{th} percentile minus 1.5 IQR (interquartile range) and 75^{th} percentile plus 1.5 IQR. The outlier values are not shown. Boxplots are derived from 50 simulations.

Table 6, below, presents a tabular summarization of results from the simulation of five methods used to measure the overall diagnostic error rates of 100 QA'd radiologists. Reported values are the median difference between the measured and actual diagnostic error rates and the 95% credible interval (CI) around the median. Summary statistics are derived from 50 simulations:

TABLE 6
Tabular summarization of simulation results of five different
diagnostic error methods; measurement of overall diagnostic
error rates of 100 QA'd radiologists.

| | Median difference (reported in | |
|---|---|---|
| Diagnostic Error | percentage points) between | |
| Rate Measurement | measured and actual diagnostic | |
| Method | error rate and 95% CI | |
| BIRAR | −0.62 | [−5.64, 4.81] |
| Perfect QA'ing | 0.00 | [−6.88, 8.14] |
| Radiologists | | |
| Single Review | 9.41 | [0.67, 18.83] |
| Majority Panel | 2.91 | [−10.60, 20.12] |
| Majority Panel with | 3.15 | [−4.95, 11.98] |
| 3x Total QA Review | | |
| Volume | | |

These results show that the presently disclosed BIRAR method is more accurate, as quantified by the median difference between the measured diagnostic error rate and the QA'd radiologist's actual diagnostic error rate, than any of the other methods tested in this study except for the “Perfect QA'ing Radiologists” method, which is only possible to evaluate in a simulation and not possible to implement in the real world. The BIRAR measurements are 80.3% more accurate than the “Majority Panel with 3× Total QA Review Volume” method (median differences between measurement of diagnostic error rate and actual diagnostic error rate of −0.62 versus 3.15 percentage points, respectively).

Further, the accuracy of the BIRAR measurements displayed lower variability than all of the other methods including the “Perfect QA'ing Radiologists” method, as quantified by 95% CI around the median difference between the QA'd Radiologists' measured and actual diagnostic error rates. The variability present in the “Perfect QA'ing Radiologists” diagnostic error rate measurements is due to the fact that a finite sample of 90 QA'd Exams per QA'd radiologist is used to calculate diagnostic error rates; the BIRAR method is able to reduce the measurement variability by 30.9% compared to what would be expected even with perfect QA'ing radiologists in the context of a QA program that aims to measure the diagnostic error rates of 100 radiologists using 9,000 total QA reviews. Similarly, the BIRAR method would reduce the measurement variability by 66.0% compared to what would be expected using the “Majority Panel” method in the context of a QA program measuring the diagnostic error rates of 100 radiologists using 9,000 total QA reviews.

FIG. 5 and Table 7 present a similar view of the study results, with the only difference being that the five measurement methods were used to calculate the “two-degree” diagnostic error rate of each QA'd radiologist. A “two-degree” diagnostic error is when the reported finding indicates that a pathology is two degrees more or less severe than it actual is (e.g., a grade 0 pathology is reported as grade 2 or vice versa). As described in the methods section, each of the QA'd radiologists had an actual two-degree diagnostic error rate of 3%.

For example, FIG. 5 is a graph 500 depicting boxplots of the difference between the measured and actual diagnostic error rates of 100 QA'd radiologists using the five error rate measurement methods simulated in this study. Standard boxplot notation is used with outliers not included in the graph. Summary statistics are derived from 50 simulations. Boxplots are derived from 50 simulations. In the context of Table 7, below, reported values are the median difference between the measured and actual diagnostic error rates and the 95% credible interval (CI) around the median. Summary statistics are derived from 50 simulations.

TABLE 7
Tabular summarization of simulation results of five different
diagnostic error methods; measurement of “two-degree”
diagnostic error rates of 100 QA'd radiologists

| | Median difference (reported in | |
|---|---|---|
| Diagnostic Error | percentage points) between | |
| Rate Measurement | measured and actual diagnostic | |
| Method | error rate and 95% CI | |
| BIRAR | 0.05 | [−1.79, 2.88] |
| Perfect QA'ing | −0.17 | [−2.67, 3.94] |
| Radiologists | | |
| Single Review | 2.25 | [−1.61, 7.35] |
| Majority Panel | 0.08 | [−2.67, 9.08] |
| Majority Panel with | 0.39 | [−2.67, 4.74] |
| 3x Total QA Review | | |
| Volume | | |

These results for the measurement of “two-degree” diagnostic errors are consistent with the results presented above for the measurement of overall diagnostic error rates. In this case, the simulation results demonstrated that the presently disclosed BIRAR method was more accurate, as quantified by the median difference between the measured diagnostic error rate and the QA'd radiologist's actual diagnostic error rate, than all of the other methods tested, including the “Perfect QA'ing Radiologists”. The BIRAR measurements are 87.2% more accurate than the “Majority Panel with 3× Total QA Review Volume” method (e.g., median differences between measurement of diagnostic error rate and actual diagnostic error rate of 0.05 versus 0.39 percentage points, respectively).

The BIRAR measurements of “two-degree” error rates also displayed lower variability than all of the other methods, as quantified by 95% CI around the median difference between the QA'd radiologists' measured and actual diagnostic error rates. The BIRAR method demonstrated a 60.3% reduction in measurement variability compared to the “Majority Panel” method in the context of a QA program that aims to measure the “two-degree” diagnostic error rates of 100 radiologists using 9,000 total QA reviews.

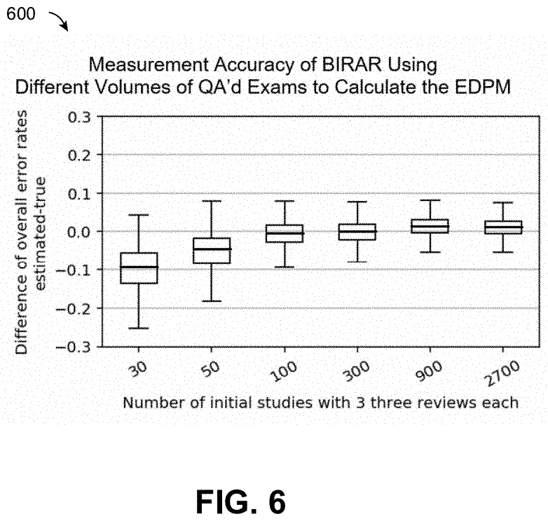

The results of the sensitivity analysis to evaluate the impact of using a different number of QA'd Exams to estimate the EDPM are depicted in the graph 600 in FIG. 6, which depicts boxplots of the difference between the BIRAR measured and actual diagnostic error rates of 100 QA'd radiologists when using different volumes of QA'd Exams to calculate the EDPM. Standard boxplot notation is used with outliers not included in the graph. The simulation results show that when keep all other configuration parameters constant, the measurement error of the BIRAR method, as quantified by the median difference between the measured diagnostic error rate and the QA'd radiologist's actual diagnostic error rate, is approximately zero when using more than 100 QA'd Exams independently reviewed three times. Modest additional reductions in the variability of the measurement error are observed up to the use of 900 QA'd Exams to calculate the EDPM:

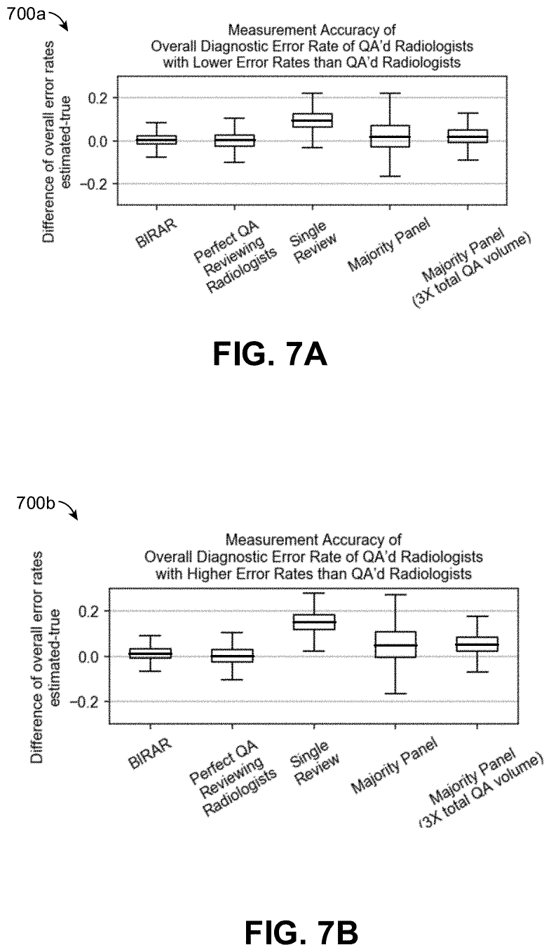

The results of the sensitivity analysis to evaluate the impact of the QA'ing radiologists' error rates on the overall study results are illustrated in FIGS. 7A and 7B, along with Table 8. FIG. 7A is a graph 700a illustrating example results of the simulation of the five methods for measuring diagnostic error rates of a population of 100 QA'd radiologists when the QA'ing radiologists all have a lower error rate than the QA'd radiologists (defined above as QA'ing Radiologist Profile 1, α^{QA'ing,1}). FIG. 7B is a graph 700b illustrating example results of the same simulation with the QA'ing radiologists' error rates all defined to be higher than the QA'd radiologists (using the QA'ing Radiologist Profile 3, α^{QA'ing,3} defined above).

More specifically, graph 700a of FIG. 7A depicts boxplots of the difference between the measured and actual diagnostic error rates of 100 QA'd radiologists using the five error rate measurement methods simulated in this study and shows that the results simulated under conditions where the QA'ing radiologists have lower error rates than QA'd radiologists. Graph 700b of FIG. 7B shows the results simulated under conditions where the QA'ing radiologists have higher error rates than QA'd radiologists. Standard boxplot notation is used with outliers not included in the graph.

Table 8 presents a tabular summarization of results from the simulation of the five methods used to measure the overall diagnostic error rates of 100 QA'd radiologists, simulated under conditions where the QA'ing radiologists have lower (middle column) and higher error (rightmost column) rates than QA'd radiologists. Reported values are the median difference between the measured and actual diagnostic error rates and the 95% credible interval (CI) around the median:

TABLE 4
Tabular summarization of simulation results of five different
diagnostic error methods; measurement of overall diagnostic
error rate when QA'ing radiologists have lower and
higher error rates than QA'd radiologists

| | Median difference (reported in percentage | | |
|---|---|---|---|
| | points) between measured and actual | | |
| Diagnostic | diagnostic error rate and 95% CI | | |
| Error Rate | | QA'ing radiologists' | QA'ing radiologists' |
| Measurement | | error rates lower | error rates higher |
| Method | | than QA'd radiologists | than QA'd radiologists |

| | | | | |
|---|---|---|---|---|
| BIRAR | 0.29 | [−5.39, 6.67] | 1.22 | [−3.75, 7.96] |
| Perfect QA'ing | 0.06 | [−7.24, 7.92] | 0.06 | [−7.24, 8.02] |
| Radiologists | | | | |
| Single Review | 9.38 | [0.84, 18.66] | 15.09 | [5.98, 24.71] |
| Majority Panel | 1.77 | [−11.24, 18.19] | 5.09 | [−9.87, 22.77] |
| Majority Panel | 2.02 | [−5.53, 10.49] | 5.21 | [−3.33, 14.53] |
| with 3x Total | | | | |
| QA Review | | | | |
| Volume | | | | |

The results of both of these sensitivity analyses show that the presently disclosed BIRAR method is more accurate, as quantified by the median difference between the measured diagnostic error rate and the QA'd radiologist's actual diagnostic error rate, than all of the other methods tested in this study except for the “Perfect QA'ing Radiologists” method. The BIRAR measurements are 85.6% more accurate than the “Majority Panel with 3× Total QA Review Volume” method when the QA'ing Radiologists have lower error rates than the QA'd Radiologists and are 76.6% more accurate when the QA'ing Radiologists have higher error rates than the QA'd radiologists.

Similarly, the accuracy of the BIRAR measurements again displayed lower variability than all of the other methods, as quantified by 95% CI around the median difference between the QA'd radiologists' measured and actual diagnostic error rates. The BIRAR method reduced the measurement variability by 20.4% compared to the “Perfect QA'ing Radiologists” method when the QA'ing radiologists have lower error rates than the QA'd radiologists and by 23.3% when the QA'ing radiologists have higher error rates than the QA'd radiologists. Compared to the “Majority Panel” method, the presently disclosed BIRAR approach demonstrated 59.0% lower variability when the QA'ing radiologists have lower error rates than the QA'd radiologists and 64.1% lower variability when the QA'ing radiologists have higher error rates than the QA'd radiologists.

### Analysis

While simulation studies can occasionally fail to capture all aspects of how a diagnostic error rate measurement methodology may work in the “real world,” it is believed that evaluating the performance of this novel BIRAR approach using simulated data was an appropriate choice because it allowed explicit and precise comparisons to be made between the diagnostic error rates calculated using various measurement methods and the actual true diagnostic error rate of hypothetical radiology exams under evaluation. A study that instead used real patient exams and QA review data would suffer from a number of complicating issues, including: uncertainty related to the gold standard used to establish ground truth diagnostic error rates, and/or the potential lack of generalizability of the study's results to other clinical contexts where diagnostic error rates and their ability to be reliably flagged through secondary assessments may vary. Efforts were made to choose realistic values to calibrate the simulation and to also investigate the sensitivity of the results to important calibration parameters, like the relative error rates of the QA'ing and QA'd radiologists.

This simulation study compared the performance of the novel BIRAR method to a “Single Review” method, which is similar the approach used for peer review processes within QA programs, and a “Majority Panel” method, which is similar to processes used in some QA programs and academic studies to measure diagnostic error rates for selected exam types. Both the “Single Review” and “Majority Panel” methods demonstrated a consistent tendency to overestimate the diagnostic error rates of the QA'd radiologists. Given the configuration of the simulation described above, the median difference between the diagnostic error rate measured using the “Single Review” method and the QA'd radiologist's actual error rate was 9.4 percentage points higher (which is a 55.4% overestimation relative to the QA'd radiologists' actual diagnostic error rate), and a 95% CI range that was 100% above zero (which means the “Single Review” measured error rate was higher than the actual error rate for essentially every QA'd radiologist). With the “Majority Panel” method, the median amount of overestimation was 2.9 percentage points (or a 17.1% overestimation of the actual diagnostic error rate), with a 95% CI range that was 65.5% above zero. The diagnostic error rate measurements using BIRAR appeared to be unbiased, with a median underestimation of 0.62 percentage points (or a 3.6% underestimation of the actual diagnostic error rate), with a 95% CI range that was more balanced around zero (54.0% above zero and 46.0% below).

As expected, the performance of all of the diagnostic error rate measurement methods was degraded when simulating a scenario in which the QA'ing radiologists had higher error rates than the QA'd radiologists. But under these conditions the “Single Review” and “Majority Panel” methods overestimated the error rates of the QA'd radiologists by a median level of 15.09 (with 95% CI of 5.98 to 24.71) and 5.09 (with 95% CI of −9.87 to 22.77) percentage points, respectively; while the presently disclosed BIRAR method achieved a median level of accuracy of 1.22 (with 95% CI of −3.75 to 7.96) percentage points.

When implementing the presently disclosed BIRAR process in order to measure the diagnostic error rates of radiologists covered by an actual QA program, standardized and structured data should be captured during QA reviews. These standardized and structured QA reviews should be customized to individual exam types so that comparable assessments about a comprehensive or curated set of the relevant finding types are made by both the QA'd and QA'ing radiologists when assessing a given exam. Separate EDPMs can then be generated for each of the selected pathology/finding types included in the structured QA reviews.

Although the presently disclosed BIRAR approach was presented in the simulation study as a two-step process in which an initial set of exams were subjected to three QA reviews to calculate the EDPM and then in a subsequent step QA'd exams were subjected to single QA reviews, various other/different implementations and/or configurations are contemplated within the scope of the disclosure. For example, in some scenarios it might be beneficial to intersperse, or periodically collect additional multiple-QA'd exams to continually update the EDPM since the underlying error rates may not be constant.

The BIRAR method is shown to be more accurate than all other methods evaluated, as quantified by median difference between measured and actual diagnostic error rates of the radiologists subject to QA review (with the exception of the hypothetical “Perfect QA-ing Radiologists” method, which is not possible in the real world given its assumption that the QA radiologists have a 0% error rate). Notably, BIRAR is demonstrated to be 80.3% more accurate than a “Majority Panel with 3× Total QA Review Volume” and additionally displayed lower accuracy variability than all other methods evaluated. Additionally, the presently disclosed BIRAR method has been shown to be a viable approach for measuring diagnostic error rates that overcomes limitations that have previously made incorporating diagnostic error rate measurements into QA programs challenging. Using BIRAR, diagnostic error rates can be measured accurately and with low variability without the need for gold standard comparison tests and without the need for multiple secondary assessments or consensus discussions for every study covered by the QA program, which allows this approach to be scaled across a large population of radiologists. Additionally, the method has been shown to be robust, still producing reliable measures of diagnostic error rates, even when the secondary QA assessments are performed by radiologists who themselves have higher error rates than the radiologists covered by the QA program.

Accordingly, BIRAR enables diagnostic error rates to be measured accurately and is shown to be robust even when QA assessments are performed by radiologists who themselves have higher error rates than the QA'd radiologists.

Machine Learning (ML) and/or Artificial Intelligence (AI) Uses

In some embodiments, the presently disclosed BIRAR-based approach can be utilized in conjunction with one or more QA'd reads and/or QA'ing reads of a radiology image that are generated by a machine learning (ML) network, artificial intelligence (AI) system, and/or various other ML and AI solutions for identifying pathologies in radiological images. In other words, the BIRAR-based QA process can, in some embodiments, be agnostic to the source of primary reads (described above as being from QA'd radiologists) and secondary reads (described above as being from QA'ing radiologists). For example, see commonly owned U.S. patent application Ser. Nos. 16/386,006; 16/849,442; and Ser. No. 16/849,506; the disclosures of which are herein incorporated by reference.

In some embodiments, primary and/or secondary QA reads of a radiological image obtained from an ML or AI solution can be commingled with the primary and secondary QA reads that are obtained from human radiologists, either with or without differentiation. In a scenario with differentiation, the computer-generated reads can be commingled with the human-generated reads, but separate EDPMs calculated for the computerized reviewers and the human radiologist reviews. Additionally or alternatively, in a scenario with differentiation, the computer-generated reads can be handled separately from human-generated reads, and the corresponding EDPM(s) separately generated.

In some embodiments, different ML or AI models can each be treated separately under the presently disclosed BIRAR-based framework, meaning that a separate EDPM is generated for each distinct ML/AI model. It is contemplated that the different ML/AI models can be provided with different architectures and/or the same architecture but a different training methodology and/or training data set.

In some embodiments, the BIRAR-based systems and methods disclosed herein can be utilized solely with computer-generated reads of radiological images, e.g., from one or more ML and/or AI systems. Additionally, feedback in terms of assessed diagnostic accuracy or error rates for one or more of the ML/AI systems can be coupled to a training process for such ML/AI systems. In some embodiments, the BIRAR accuracy/error feedback can be coupled to a re-training process for the existing ML/AI models that are providing the computer-generated reads of radiological images. For example, if a given ML/AI model participating in the BIRAR-based QA process is determined to have fallen below a minimum or otherwise pre-determined diagnostic error rate threshold, this can automatically trigger a re-training process for the given ML/AI model. In some embodiments, such a re-training process can be augmented by the BIRAR-based QA process, for example by identifying the particular types or classifications of radiological images that were diagnosed incorrectly, identifying the types of most common or problematic errors made, etc., and using this identified information to generate a customized re-training data set designed to specifically address the assessed shortcomings of the ML/AI model as determined by the BIRAR-based QA process.

Additionally, in some embodiments the presently disclosed BIRAR-based analytical framework, systems and methods can be applied to domains other than QA processes for radiological image review (e.g., the primary example discussed above). For example, the presently disclosed BIRAR-based approach can be utilized in the correction of noisy classifier outputs from a wide variety of different ML/AI systems, models, etc. In some embodiments, the BIRAR-based approach can be utilized in conjunction with ML/AI training and/or validation processes in order to improve the ultimate classification performance of the trained system.

### APPENDIX A

Example Stan implementation of model given in Eq. (6)

```
data {
  int<lower=1> N_studies;
  int<lower=1> N_reviews;
  int<lower=1> N_grades;
  int<lower=1,upper=N_grades> r[N_studies,N_reviews];
  int<lower=1,upper=N_grades> o[N_studies,1];
}
transformed data {
  vector[N_grades] alpha_p_qad = rep_vector(1,N_grades);
  vector[N_grades] alpha_p_qaing = rep_vector(1,N_grades);
  vector[N_grades] alpha_p_s = rep_vector(1,N_grades);
}
parameters {
  simplex[N_grades] p_s;
  simplex[N_grades] p_qad[N_grades];
  simplex[N_grades] p_qaing[N_grades];
}
model {
  vector[N_grades] tmp;
  p_s ~ dirichlet(alpha_p_s);
  for (j in 1:N_grades) {
    p_qad[j] ~ dirichlet(alpha_p_qad);
    p_qaing[j] ~ dirichlet(alpha_p_qaing);
  }
  for (i in 1:N_studies) {
    for (j in 1:N_grades) {
      tmp[j] = log(p_s[j])+categorical_lpmf(o[i]|p_qad[j])
        +categorical_lpmf(r[i]|p_qaing[j]);
    }
    target += log_sum_exp(tmp);
  }
}
```

### APPENDIX B

### Generative Model

### Generative Model

### Definition

Next, we formulate a hierarchical model for o as follows

p^{QA'd}|α^{QA'd}˜Dirichlet(α^{QA'd}),

o|p^{QA'd}˜Categorical(p^{QA'd}), (3)

where α^{QA'd} is a user-defined parameter.

Similarly, we formulate a hierarchical model for r as follows

p^{QA'ing}|α^{QA'ing}˜Dirichlet(α^{QA'ing}),

r|p^{QA'ing}˜Categorical(p^{QA'ing}), (4)

where α^{QA'ing} is a user-defined parameter.

We consider N_{grades} patient profiles. Therefore, we formulate a hierarchical model for patient profile, s∈0, 1, . . . , N_{grades}−1, as follows

s|p^{s}˜Categorical(p^{s}) (5)

where p^{s} is a user-defined parameter.

Next, by combining the previous models, we can formulate a hierarchical generative model for y as follows

sᵢ|p^{s}˜Categorical(p^{s}),i=1,2, . . . ,N_{studies},

pᵢ^{QA'd}|αₛ_{ᵢ}^{QA'd}˜Dirichlet(αₛ_{ᵢ}^{QA'd}),i=1,2, . . . ,N_{studies},

oᵢ|pᵢ^{QA'd}˜Categorical(pᵢ^{QA'd}),i=1,2, . . . ,N_{studies},

mᵢ=Permutate((1,2, . . . ,N_{QA'ing radiologists})),

p_{i,j}^{QA'ing}|αₛ_{ᵢ}^{QA'ing,m}^{_{i,j}}˜Dirichlet(αₛ_{ᵢ}^{QA'ing,m}^{_{i,j}}),i=1,2, . . . ,N_{studies},j=1,2, . . . ,N_{reviewers},

r_{i,j}|p_{i,j}^{QA'ing}˜Categorical(p_{i,j}^{QA'ing}),i=1,2, . . . ,N_{studies},j=1,2 . . . ,N_{reviewers},

y_{i,j}=ƒ(oᵢ,r_{i,j}),i=1,2, . . . ,N_{studies},j=1,2, . . . ,N_{reviewers}. Eq. (6)

Note that m is used to permutate the order of QA'ing Radiologists for each study separately. This is needed so that in the case of N_{reviews} less than the number of QA'ing Radiologists, the studies are not always reviewed by the same subset of QA'ing Radiologists.

### Implemention

Forward simulation of the model defined in Equation (7) can be implemented in Stan as shown in Listing 1. This implementation encodes the discrepancy values as integers.

Listing 1: A Stan implemention of the model defined in Equation (6)

```
functions {
  int[ ] permutation_rng(int N) {
          int y[N];
          for (n in 1:N)
            y[n] = n;
          vector[N] theta = rep_vector(1.0/N,N);
          for (n in 1:N) {
            int i = categorical_rng(theta);
            int tmp = y[n];
            y[n] = y[i];
            y[i] = tmp;
          }
          return y;
      }
      int f(int o, int r) {
        if (o+1 == r) return 1; // 1-degree undercall
        else if (o−1 == r) return 2; // 1-degree overcall
        else if (o+1 < r) return 3; // 2-degree undercall
        else if (o−1 > r) return 4; // 2-degree overcall
        else return 0; // Agree
      }
    }
    data {
      int N_grades;
      int N_studies;
      int N_reviewers;
      int N_reviews;
      vector<lower=0>[N_grades] alpha_qad[N_grades];
      vector<lower=0>[N_grades] alpha_qaing[N_reviewers,N_grades];
      simplex[N_grades] p_s;
    }
generated quantities {
  int<lower=0,upper=N_grades−1> o[N_studies];
  int<lower=0,upper=N_grades−1> r[N_studies,N_reviews];
  int<lower=0,upper=N_grades−1> s[N_studies];
  int<lower=1,upper=N_reviewers> m[N_studies,N_reviewers];
  int<lower=0,upper=4> y[N_studies,N_reviews];
  simplex[N_grades] p_qad[N_studies];
  simplex[N_grades] p_qaing[N_studies,N_reviews];
  for (i in 1:N_studies) {
    s[i] = categorical_rng(p_s)−1;
    p_qad[i] = dirichlet_rng(alpha_qad[s[i]+1]);
    o[i] = categorical_rng(p_qad[i])−1;
    m[i] = permutation_rng(N_reviewers);
    for (j in 1:N_reviews) {
          p_qaing[i,j] = dirichlet_rng(alpha_qaing[m[i,j],s[i]+1]);
          r[i,j] = categorical_rng(p_qaing[i,j])−1;
          y[i,j] = f(o[i],r[i,j]);
      }
    }
  }
```

What is claimed is:

1. A method comprising:
obtaining an initial set of diagnostic imaging exams, wherein each diagnostic imaging exam includes a severity grade associated with an initial radiologist;
for each diagnostic imaging exam of the initial set, obtaining two or more secondary quality assurance (QA) reviews for each respective diagnostic imaging exam, wherein the secondary QA reviews are associated with one or more QA'ing radiologists different than the initial radiologist;
determining one or more inter-reviewer agreement rates for the QA'ing radiologists, based at least in part on the secondary QA reviews associated with the QA'ing radiologists; and
determining a diagnostic error associated with one or more initial radiologists, wherein the diagnostic error is determined based at least in part on the one or more inter-reviewer agreement rates for the QA'ing radiologists and a subsequent diagnostic imaging exam obtained for a respective one of the initial radiologists.

2. The method of claim 1, wherein determining the one or more inter-reviewer agreement rates comprises generating one or more Error Detection Probability Matrices (EDPMs) for the QA'ing radiologists.

3. The method of claim 2, wherein the one or more EDPMs are generated based on an initialization data set, the initialization data set including the initial set of diagnostic imaging exams and the two or more secondary QA reviews obtained for each respective diagnostic imaging exam of the initial set.

4. The method of claim 2, further comprising:
generating an EDPM for each QA'ing radiologist associated with the initial set of diagnostic imaging exams, wherein at least one secondary QA review included in the initialization data set is obtained from each QA'ing radiologist.

5. The method of claim 4, wherein generating the EDPM for each QA'ing radiologist includes:
determining one or more conditional probabilities that a diagnostic error of a specific type is present in a given diagnostic imaging exam included in the initial set of diagnostic imaging exams;
wherein each respective conditional probability is determined given a presence of an identified discrepancy type determined from the secondary QA reviews included in the initialization data set and associated with the QA'ing radiologist.

6. The method of claim 4, wherein:
generating the EDPM for each QA'ing radiologist further includes determining a discrepancy value for each secondary QA review associated with the QA'ing radiologist; and
the discrepancy value is determined based on analyzing a severity grade associated with a given secondary QA review associated with the QA'ing radiologist and the corresponding severity grade associated with the initial radiologist, wherein both severity grades are associated with the same diagnostic imaging exam of the initial set of diagnostic imaging exams.

7. The method of claim 6, wherein generating the EDPM for each QA'ing radiologist further includes determining an error detection probability for each respective QA'ing radiologist, wherein:
the error detection probability is based on one or more conditional probability distributions over a set of possible severity grades; and
the one or more conditional probability distributions are determined given the severity grade associated with the given secondary QA review from the QA'ing radiologist and the corresponding severity grade associated with the initial radiologist.

8. The method of claim 7, further comprising determining the one or more conditional probability distributions using a hierarchical generative model for the discrepancy values, wherein the one or more conditional probability distributions are modeled as Dirichlet distributions.

9. The method of claim 8, further comprising utilizing the Dirichlet distributions as hierarchical priors for determining the one or more conditional probability distributions.

10. The method of claim 1, wherein the initial set of diagnostic imaging exams includes 300-500 different diagnostic imaging exams

11. The method of claim 2, wherein the one or more EDPMs are generated for a group of QA'ing radiologists or a subset of the group of QA'ing radiologists.

12. An apparatus comprising:
at least one memory; and
at least one processor coupled to the at least one memory, the at least one processor configured to:
obtain an initial set of diagnostic imaging exams, wherein each diagnostic imaging exam includes a severity grade associated with an initial radiologist;
for each diagnostic imaging exam of the initial set, obtain two or more secondary quality assurance (QA) reviews for each respective diagnostic imaging exam, wherein the secondary QA reviews are associated with one or more QA'ing radiologists different than the initial radiologist;
determine one or more inter-reviewer agreement rates for the QA'ing radiologists, based at least in part on the secondary QA reviews associated with the QA'ing radiologists; and
determine a diagnostic error associated with one or more initial radiologists, wherein the diagnostic error is determined based at least in part on the one or more inter-reviewer agreement rates for the QA'ing radiologists and a subsequent diagnostic imaging exam obtained for a respective one of the initial radiologists.

13. The apparatus of claim 12, wherein to determine the one or more inter-reviewer agreement rates, the at least one processor is configured to generate one or more Error Detection Probability Matrices (EDPMs) for the QA'ing radiologists.

14. The apparatus of claim 13, wherein the one or more EDPMs are generated based on an initialization data set, the initialization data set including the initial set of diagnostic imaging exams and the two or more secondary QA reviews obtained for each respective diagnostic imaging exam of the initial set.

15. The apparatus of claim 13, wherein the at least one processor is further configured to:
generate an EDPM for each QA'ing radiologist associated with the initial set of diagnostic imaging exams, wherein at least one secondary QA review included in the initialization data set is obtained from each QA'ing radiologist.

16. The apparatus of claim 15, wherein to generate the EDPM for each QA'ing radiologist, the at least one processor is configured to:
determine one or more conditional probabilities that a diagnostic error of a specific type is present in a given diagnostic imaging exam included in the initial set of diagnostic imaging exams;
wherein each respective conditional probability is determined given a presence of an identified discrepancy type determined from the secondary QA reviews included in the initialization data set and associated with the QA'ing radiologist.

17. The apparatus of claim 15, wherein:
to generate the EDPM for each QA'ing radiologist, the at least one processor is further configured to determine a discrepancy value for each secondary QA review associated with the QA'ing radiologist; and
the discrepancy value is determined based on analyzing a severity grade associated with a given secondary QA review associated with the QA'ing radiologist and the corresponding severity grade associated with the initial radiologist, wherein both severity grades are associated with the same diagnostic imaging exam of the initial set of diagnostic imaging exams.

18. The apparatus of claim 17, wherein to generate the EDPM for each QA'ing radiologist, the at least one processor is further configured to determine an error detection probability for each respective QA'ing radiologist, wherein:
the error detection probability is based on one or more conditional probability distributions over a set of possible severity grades; and
the one or more conditional probability distributions are determined given the severity grade associated with the given secondary QA review from the QA'ing radiologist and the corresponding severity grade associated with the initial radiologist.

19. The apparatus of claim 18, wherein the at least one processor is further configured to determine the one or more conditional probability distributions using a hierarchical generative model for the discrepancy values, wherein the one or more conditional probability distributions are modeled as Dirichlet distributions.

20. The apparatus of claim 19, wherein the at least one processor is further configured to utilize the Dirichlet distributions as hierarchical priors for determining the one or more conditional probability distributions.
